(19) United States
(12) Patent Application Publication
(10) Pub. No.: US 2007/0238864 A1
(43) Pub. Date: Oct. 11, 2007

(54) MULTIDIMENSIONAL PROTEIN SEPARATION

(75) Inventors: Andrew K. Ottens, Gainesville, FL (US); Firas Hosni Kobeissy, Gainesville, FL (US); Nancy D. Denslow, Gainesville, FL (US); Kevin K. Wang, Gainesville, FL (US)

Correspondence Address: AKERMAN SENTERFITT, P.O. BOX 3188, WEST PALM BEACH, FL, 33402-3188, US

(73) Assignee: University of Florida Research Foundation, Inc., Gainesville, FL (US)

(21) Appl. No.: 11/551,141

(22) Filed: Oct. 19, 2006

Related U.S. Application Data
(63) Continuation-in-part of application No. PCT/US05/13016, filed on Apr. 19, 2005.
(60) Provisional application No. 60/563,396, filed on Apr. 19, 2004.

(51) Int. Cl. C07K 1/16

(52) U.S. Cl. 530/413

(57) ABSTRACT

In large scale proteome applications, protein separation is paramount to observing discrete changes and quantitative evaluation must coincide with qualitative protein identification for effective differential analysis. A four dimensional (4D) platform for resolving and differentially analyzing complex biological samples is presented. The system, collectively termed CAX-PAGE/RPLC-MSMS, combines bi-phasic ion-exchange chromatography (1^{st} dimension) and polyacrylamide gel electrophoresis (2^{nd} dimension) for protein separation, quantification and differential band targeting leading toward subsequent capillary reverse phase liquid chromatography (3^{rd} dimension) and data dependant tandem mass spectrometry (4^{th} dimension) for semi-quantitative and qualitative peptide analysis.

### CROSS-REFERENCE TO RELATED APPLICATION

The present application is a continuation-in-part of PCT application number PCT/US2005/013016, entitled “MULTIDIMENSIONAL PROTEIN SEPARATION” filed Apr. 19, 2005, which claims priority to U.S. provisional application No. 60/563,396, entitled “COMBINED CATIONIC ANIONIC EXCHANGE TANDEM GEL ELECTROPHORESIS PROTEIN SEPARATION,” filed Apr. 19, 2004, which are both incorporated herein by reference in their entirety.

### STATEMENT REGARDING FEDERALLY SPONSORED RESEARCH OR DEVELOPMENT

The United States Government may have certain rights in this invention pursuant to the Department of Defense Contract No. DAMD17-03-1-0066.

### FIELD OF THE INVENTION

The invention relates to the field of proteomics. In particular, a system and methods for identification and quantification of proteins and peptides from complex biological samples is provided.

### BACKGROUND OF INVENTION

From the recent completion of human and other species genomes it has become apparent that many biological systems operate through changes at the protein level not governed by gene regulation (Denslow N et al., J Neurotrauma (2003) 20, 401-407). The new field of proteomics has arisen to provide a more complete picture of cell operation at the protein level under normal and challenged conditions. The pervasive influence of proteomic technology has been rapid, however many challenges still persist. Of primary concern is the inherent complexity of biological protein mixtures: the shear number of proteins (10,000 or more) and the wide dynamic range of concentration for example. To handle this challenge, most research laboratories have relied on two-dimensional polyacrylamide gel electrophoresis (2D-PAGE) for protein separation (Griffin T. J. et al. J Bio. Chem. (2001) 276, 45497-45500. Peng J. et al. J Mass Spectrom. (2001) 36, 1083-1091). There are numerous limitations to this technology that have prompted researchers to look elsewhere in particular difficulties with gel-to-gel reproducibility, dynamic range, pI range, the ability to resolve very small and large proteins and those that are hydrophobic in nature restrict the use of 2D-PAGE.

The use of ion-exchange has been limited to that of a pre-fractionation step for separating a particular group or proteins, i.e., a clean-up method for analysis of a subset of proteins from mixtures. Ion-exchange has also been incorporated prior to reverse phase separations for peptide analysis post enzymatic digestion. This typically incorporates an acidic modifier to shift the charge distribution to allow more peptides to adhere to the ion-exchange support. In any case, a large number of proteins or peptides from a complex mixture are not retained by ion-exchange columns as they are either opposite to or neutral in charge relative to proper operating conditions.

An urgent need thus exists in the art for the to separate, identify and purify complex biological mixtures.

### SUMMARY

Differential proteomic analysis has arisen as a large scale means to discern proteome wide changes upon treatment, injury or disease. In large scale proteome applications, protein separation is paramount to observing discrete changes. In addition, quantitative evaluation must coincide with qualitative protein identification for effective differential analysis. A four dimensional (4D) platform for resolving and differentially analyzing complex biological samples is presented.

In a preferred embodiment, separation and differential analysis of proteins and/or peptides in a crude biological sample comprises a method based on four independent physical properties and two complimentary quantification methods are employed. The platform, collectively termed CAX-PAGE/RPLC-MSMS, combines bi-phasic ion-exchange chromatography (1^{st} dimension) and polyacrylamide gel electrophoresis (2^{nd} dimension) for protein separation, quantification and differential band targeting leading toward subsequent capillary reverse phase liquid chromatography (3^{rd} dimension) and data dependant tandem mass spectrometry (4^{th} dimension) for semi-quantitative and qualitative peptide analysis.

In another preferred embodiment, a method of isolating and quantifying biomarkers, comprises obtaining a crude biological sample; subjecting the sample to a bi-phasic ion-exchange chromatography and obtaining fractions; separating the fractions by polyacrylamide gel electrophoresis into bands according to molecular weight; cutting bands from the polyacrylamide gel; subjecting the separated bands to capillary reverse phase liquid chromatography and obtaining a second set of fractions; and, subjecting the second set of fractions to tandem mass spectrometry; thereby, isolating and quantifying the isolated biomarkers. The fractions can be collected in a plate format, such as a 96-well plate, to further expedite the analysis.

In another preferred embodiment, a method of isolating, quantifying biomarkers comprises obtaining a crude biological sample(s); clarifying the sample(s) via centrifuigation and ultrafiltration; subjecting the samples sequentially to bi-phasic ion-exchange chromatography and obtaining fractions; separating fractions by polyacrylamide gel electrophoresis into bands according to molecular weight and quantitatively imaging band density and evaluating protein expression; cutting selected bands from the polyacrylamide gel and subjecting them to in-gel digestion; subjecting the digested bands to capillary reverse phase liquid chromatography in tandem with mass spectrometry; thereby, isolating, quantifying and identifying the biomarker associated peptides. The biomarkers can be collected into 96-well plates or other formats and analysis can be conducted with any automated means or semi-automated means, if the user so desires.

In another preferred embodiment, the ion-exchange chromatography comprises at least a plurality of gradients, preferably, the ion exchange chromatography comprises at least a two step gradient, preferably, the ion exchange chromatography comprises a three step gradient, preferably, the ion exchange chromatography comprises a five step gradient, preferably, ion exchange chromatography comprises a ten step gradient, preferably, the ion exchange chromatography comprises between about a two step gradient up to a twenty step gradient.

In another preferred embodiment, the ion-exchange chromatography comprises a plurality of ion exchange media. Preferably, the media comprises weak anionic and/or cationic exchange media and strong anionic and/or cationic media.

In another preferred embodiment, the bi-phasic ion ion-exchange chromatography comprises at least a two step gradient, preferably the bi-phasic ion exchange chromatography comprises a three step gradient. Two step gradient comprise linear transitions from 0% to about 15% in a volume of about 12 mL. Three step gradients comprise a linear transition from about 15% to about 50% in a volume of about 7 mL, held at about 50% in a volume of about 2 mL and re-equilibrated to 0% in about 1 mL volume.

In another preferred embodiment, the two-step gradient comprises a linear transition from 0% to about 15% in a volume of about 12 mL up to 50 mL.

In another preferred embodiment, the three-step gradient comprises a linear transition from about 15% to about 50% in a volume of about 7 mL up to 50 mL, held at about 50% in a volume of about 2 mL up to 50 mL and re-equilibrated to 0% in about 1 mL up to 50 mL volume.

In accordance with the invention, the gradient is optimized depending on the viscosity of the mixture, the complexity of the biological sample and the like and can include a plurality of gradients.

In another preferred embodiment, the polyacrylamide gel comprises a gradient of between about 1% up to 50% and/or can be a gel without a gradient. The percentage of the gel can be from about 1% to about 50%.

In accordance with the invention, the bands on the gel can be visualized using any number of dyes. For example, Coomassie blue, silver staining, Sypro Ruby, cyanine dyes and the like.

In a preferred embodiment, the bands are subjected to enzymatic digestion in-gel. Alternatively, the bands are excised and subjected to enzymatic digestion. The preferred enzymes include, but not limited to hydrolases—these include esterases, carbohydrases, nucleases, deaminases, amidases, and proteases; Hydrases such as fumarase, enolase, aconitase and carbonic anhydrase; oxidases, dehydrogenases; transglycosidases; transphosphorylases and phosphomutases; transaminases; transmethylases; transacetylases; desmolases; isomerases; ligases. Preferably, the enzyme is a tryptase.

In another preferred embodiment, the enzyme digested bands are subjected to reverse phase liquid chromatography. Preferably, the n_{c} values of the reverse phase liquid chromatography are between about 100 to about 250.

In another preferred embodiment, the fractions eluted from the reverse phase liquid chromatography are further subjected to tandem mass spectrometry and separated by mass-to-charge. Preferably, the n_{c} values are at least about 1×10⁵ up to 1×10¹⁰.

In another preferred embodiment, a method of isolating and quantifying proteins and/or peptides comprises obtaining a crude biological sample(s); clarifying the sample(s) via centrifugation and ultrafiltration; subjecting the samples sequentially to bi-phasic ion-exchange chromatography and obtaining fractions; separating fractions by polyacrylamide gel electrophoresis into bands according to molecular weight and quantitatively imaging band density and evaluating protein expression; cutting selected bands from the polyacrylamide gel and subjecting them to in-gel digestion; subjecting the digested bands to capillary reverse phase liquid chromatography in tandem with mass spectrometry; thereby, isolating, quantifying and identifying the peptides.

In another preferred embodiment, the ion-exchange chromatography comprises at least a plurality of gradients, preferably, the ion exchange chromatography comprises at least a two step gradient, preferably, the ion exchange chromatography comprises a three step gradient, preferably, the ion exchange chromatography comprises a five step gradient, preferably, ion exchange chromatography comprises a ten step gradient, preferably, the ion exchange chromatography comprises between about a two step gradient up to a twenty step gradient.

In another preferred embodiment, the ion-exchange chromatography comprises a plurality of ion exchange media. Preferably, the media comprises weak anionic and/or cationic exchange media and strong anionic and/or cationic media, for example Waters Protein Pak, Pharmacia's Source Q, etc.

In another preferred embodiment, the bi-phasic ion ion-exchange chromatography comprises at least a two step gradient, preferably the bi-phasic ion exchange chromatography comprises a three step gradient. Two step gradient comprise linear transitions from 0% to about 15% in a volume of about 12 mL. Three step gradients comprise a linear transition from about 15% to about 50% in a volume of about 7 mL, held at about 50% in a volume of about 2 mL and re-equilibrated to 0% in about 1 mL volume.

In another preferred embodiment, the two-step gradient comprises a linear transition from 0% to about 15% in a volume of about 12 mL up to 50 mL.

In another preferred embodiment, the three-step gradient comprises a linear transition from about 15% to about 50% in a volume of about 7 mL up to 50 mL, held at about 50% in a volume of about 2 mL up to 50 mL and re-equilibrated to 0% in about 1 mL up to 50 mL volume.

In another preferred embodiment, the bi-phasic ion ion-exchange chromatography comprises at least a plurality of gradients, preferably, the bi-phasic ion exchange chromatography comprises at least a two step gradient, preferably, the bi-phasic ion exchange chromatography comprises a three step gradient, preferably, the bi-phasic ion exchange chromatography comprises a five step gradient, preferably, bi-phasic ion exchange chromatography comprises a ten step gradient, preferably, the bi-phasic ion exchange chromatography comprises between about a two step gradient up to a twenty step gradient.

In accordance with the invention, the gradient is optimized depending on the viscosity of the mixture, the complexity of the biological sample and the like and can include a plurality of gradients.

In another preferred embodiment, the polyacrylamide gel comprises a gradient of between about 1% up to 50% and/or can be a gel without a gradient. The percentage of the gel can be from about 1% to about 50%.

In accordance with the invention, the bands on the gel can be visualized using any number of dyes. For example, Coomassie blue, silver staining, Sypro Ruby, cyanine dyes and the like.

In a preferred embodiment, the bands are subjected to enzymatic digestion in-gel. Alternatively, the bands are excised and subjected to enzymatic digestion. The preferred enzymes include, but not limited to hydrolases—these include esterases, carbohydrases, nucleases, deaminases, amidases, and proteases; Hydrases such as fumarase, enolase, aconitase and carbonic anhydrase; oxidases, dehydrogenases; transglycosidases; transphosphorylases and phosphomutases; transaminases; transmethylases; transacetylases; desmolases; isomerases; ligases. Preferably, the enzyme is a tryptase.

In another preferred embodiment, the enzyme digested bands are subjected to reverse phase liquid chromatography. Preferably, the n_{c} values of the reverse phase liquid chromatography are between about 100 to about 250.

In another preferred embodiment, the fractions eluted from the reverse phase liquid chromatography are further subjected to tandem mass spectrometry and separated by mass-to-charge. Preferably, the n_{c} values are at least about 1×10⁵ up to 1×10¹⁰.

Accordingly, in one embodiment, the subject invention pertains to a method of identifying at least one biomarker comprising obtaining a biological sample from a patient known to have an injury, disorder or pathological condition (test sample(s)); obtaining at least one biological sample from a patient known not to have such injury or pathological condition (control sample(s)); sequentially performing CAX chromatography to said biological samples to produce fraction samples; subjecting fraction samples to electrophoresis in a gel; visualizing proteins in said gel; identifying presence of proteins in one sample not present in another sample, wherein differential presence indicates a biomarker candidate. Preferably, subjecting fraction samples to electrophoresis comprises performing 1-D PAGE. Also preferred is running electrophoresis with fractions from the test sample side-by-side with corresponding fractions from the control sample. Visualizing the proteins may comprise staining fractions from the control sample with a first dye and staining fractions from the test sample with a different dye. The corresponding fraction samples may be overlaid whereby different colors generated indicate the presence of a protein in one or the other sample, or both. The method of identifying biomarkers can be applied to identify biomarkers relating to, but not limited to neurological injuries, disorders and diseases; cancer; autoimmune disorders; stress; exposure to toxins; and joint disease. In the case of identifying biomarkers for brain injury, blood, serum or central spinal fluid samples from individuals known to have a brain injury are compared to individuals known not to have a brain injury. Generally speaking, the sample may be tissue homogenate, urine, blood, CSF, serum or other biological fluid present in the body.

In a preferred embodiment, a method of isolating and differential quantitative analysis of proteins and/or peptides in complex biological mixtures, said method comprising: obtaining a crude biological sample; subjecting the sample to a bi-phasic ion-exchange chromatography and obtaining fractions; running the fractions obtained in order of elution side-by-side on a polyacrylamide gel electrophoresis allowing for differential comparison; quantifying bands obtained by polyacrylamide gel electrophoresis by densitometric scanning; selecting bands which are differentially expressed at least about two-fold as compared to a normal control; digesting the differentially expressed bands with enzyme; subjecting the enzyme digested bands to capillary reverse phase liquid chromatography online in tandem with mass spectrometry; thereby, isolating and quantifying the isolated proteins and/or peptides. Preferably, quantification of isolated proteins is validated by comparing the protein amounts with gel band density.

In another preferred embodiment, the bi-phasic ion ion-exchange chromatography comprises at least a plurality of gradients, preferably, the bi-phasic ion exchange chromatography comprises at least a two step gradient, preferably, the bi-phasic ion exchange chromatography comprises a three step gradient, preferably, the bi-phasic ion exchange chromatography comprises a five step gradient, preferably, bi-phasic ion exchange chromatography comprises a ten step gradient, preferably, the bi-phasic ion exchange chromatography comprises between about a two step gradient up to a twenty step gradient.

In accordance with the invention, the gradient is optimized depending on the viscosity of the mixture, the complexity of the biological sample and the like and can include a plurality of gradients.

In another preferred embodiment, the polyacrylamide gel comprises a gradient of between about 1% up to 50% and/or can be a gel without a gradient. The percentage of the gel can be from about 1% to about 50%.

In accordance with the invention, the bands on the gel can be visualized using any number of dyes. For example, Coomassie blue, silver staining, Sypro Ruby, cyanine dyes and the like.

In another preferred embodiment, bands on the gel digested by enzymes selected from the group consisting of hydrolases, esterases, carbohydrases, nucleases, deaminases, amidases, proteases, hydrases, fumarase, enolase, aconitase carbonic anhydrase, oxidases, dehydrogenases; transglycosidases; transphosphorylases phosphomutases, transaminases; transmethylases, transacetylases, desmolases, isomerases; and ligases. Preferably, the enzyme is a tryptase.

In another preferred embodiment, the enzyme digested bands are subjected to reverse phase liquid chromatography. Preferably, the n_{c} values of the reverse phase liquid chromatography are between about 100 to about 250.

In another preferred embodiment, the fractions eluted from the reverse phase liquid chromatography directly flow into the mass spectrometry and separated by mass-to-charge. Preferably, the n_{c} values are at least about 1×10⁵, preferably, the n_{c} values are about 1×10⁶, preferably, the n_{c} values are about 1×10⁷, preferably, the n_{c} values are about 1×10⁸, preferably, the n_{c} values are about 1×10⁹, preferably, the n_{c} values are about 1×10¹⁰.

Other aspects of the invention are described infra.

### BRIEF DESCRIPTION OF DRAWINGS

The invention is pointed out with particularity in the appended claims. The above and further advantages of this invention may be better understood by referring to the following description taken in conjunction with the accompanying drawings, in which:

FIG. 1 shows ion-exchange chromatograms of 1 mg of rat cerebellum brain tissue lysate separated with a NaCl gradient: strong-cationic ion-exchange (SCX) separation of tissue lysate; strong-anionic ion-exchange (SAX) separation of tissue lysate; tandem cationic and anionic ion-exchange (CAX) separation of tissue lysate. Timing of the two stage gradient is as indicated.

FIG. 2 shows a rat cerebellum proteome visualized on 1D-PAGE following CAX fractionation. MKR indicates molecular weight markers.

FIG. 3A shows a chromatogram of rat cerebellum brain lysate (1 mg protein) run sequentially in triplicate by CAX.

FIG. 3B is a gel showing selected fractions (paired as indicated) from three replicate CAX runs resolved and visualized side-by-side on 1D-PAGE. Protein compliment remained constant while band intensity varied on average by only 6%.

FIG. 4 shows a chromatogram of rat cerebellum tissue lysate (750 μg) performed with SCX, SAX, and CAX with two step elution processes.

FIGS. 5A-5B shows a comparison of rat cerebellum and cortex proteomes via sequential CAX and side-by-side 1D-PAGE. FIG. 5A is a chromatogram showing an overlay of cerebellum and cortex CAX chromatograms at 280 nm. FIG. 5B shows a side-by-side pairing of 25 fractions run on 1D-PAGE. (M=cerebellum; X=cortex). Boxed bands were excised for protein identification. Note letter labeling for correlation with Tables 1 and 2.

FIG. 6 shows a colorized rat cerebellum-cortex differential proteome display after CAX-PAGE. The colorized display was performed by overlaying adjacent lanes from FIG. 5B.

FIGS. 7A and 7B show 2D-DIGE differential display of rat cerebellum-cortex. FIG. 7A is a false color overlay of cortex Cy3 (green) and cerebellum Cy5 (red) labeled DIGE images. FIG. 7B shows the results of 2D differential software analysis comparing cortex and cerebellum tissue. Spots with 100% difference between samples are indicated by yellow for greater in cortex and green for greater in cerebellum, while blue indicates spots found only in one sample.

FIGS. 8A-8D are scans of blots showing high-throughput immunoblotting (HTPI) of liver samples using a custom 40 antibody miniscreen array. Liver tissue obtained from four rats in each experimental group was pooled and processed as described in the Materials and methods in detail. FIG. 8A, Control rat livers; FIG. 8B, rat livers subjected to 30 min of normothermic ischemia followed by 30 min of reperfusion (I/R, 30/30); and FIG. 8C, 8D, control rat livers treated with recombinant caspase-3 (C) or calpain-2 (D) in vitro. Representative blot from two runs of identical samples is shown. Squires depict proteins up or down regulated in I/R, caspase-3- and calpain-2-treated livers versus control. The numbers indicate the lane number on the screen. Lanes: 2, nitric oxide synthase (nNOS); 8, arginase-I; 9, squalene synthase (SQS); 12, β-catenin; 13, α-actinin; 16, MEK5; 27, ASS; 31, ninjurin. ASS-BDP, ASS breakdown products; MEK5-BDP, MEK5 breakdown products.

FIG. 9 is a scan of gels showing differential SDS-PAGE display of protein fractions collected after combined cation-anion exchange (CAX) chromatography. The CAX fractions obtained from control (C) and I/R (T) samples were paired up and loaded side-by-side on SDS-PAGE. Proteins with differential expression were quantified using Phoretix 1D software. The numbers represent fraction number of control (C) or I/R sample, respectively. Portions of two gels containing fractions 1-15 are shown. Labeled boxes depict differentially displayed proteins already identified by RPLC-MS-MS. Proteins identity determined by RPLC-MS-MS is shown close to the red boxes. Unlabeled boxes indicate proteins to be identified. EST-1, oestrogene preferring sulfotransferase 1E; CSP-1, carbarnoyl phosphate synthase 1; ACL, ATP citrate lyase; GRP, glucose-regulated protein p58.

FIGS. 10A-D are scans of gels hepatic expression of cytoskeletal αII-spectrin, liver-specific marker proteins, and their breakdown products following liver ischemia/reperfusion injury. Liver samples were obtained from intact animals, control (sham operated) or rats subjected to 30 min hepatic ischemia (I/R) followed by 10- and 30 min reperfusion. Intact liver tissue lysates were treated in vitro with recombinant caspase-3 or calpain-2. FIG. 10A as described in the Materials and methods in detail. Hepatic proteins (25 mg) were analyzed by SDS-PAGE/Western blotting with antibodies to non-erythroid αII-spectrin (FIG. 10A), argininosuccinate synthase (FIG. 10B), arginase-I (FIG. 10C) and estrogen sulfotransferase (FIG. 10D), and visualized using enhanced chemiluminescence (ECL). FIG. 10A shows accumulation of αII-spectrin breakdown products in I/R livers similar to caspase-3 (120 kDa) and calpain-2 (145 kDa)-dependent cleavage fragments; FIG. 10B, appearance of caspase-3-dependent ASS cleavage fragments within 10 min after reperfusion; and FIGS. 10C, 10D, hepatic levels of arginase-I and EST-1 after 10 and 30 min of reperfusion. Representative western blot images from three different caspase-3 and calpain-2 treatments of pooled intact liver tissues (FIG. 10A) and from four experimental rats in each group of I/R injury are shown.

FIGS. 11A and 11B are scans of immunoblots showing accumulation of biomarkers of liver injury in blood after hepatic ischemia/reperfusion, chronic alcoholic disease and acute endotoxic liver injury. FIG. 11A: Blood was withdrawn from rat heart after 30 min of ischemia followed by 30 min of reperfusion and from chronic alcoholic rats as described in the Materials and methods. FIG. 11B: Rats were treated with LPS/D-galactosamine or saline as described in the Materials and methods. Serum or plasma was collected and equal volumes (10 ml) were processed as described in the Materials and methods in detail. Proteins were separated by SDS-PAGE and immunoblotted with antibody against ASS, EST-1 or alanine aminotransferase ALT. Membranes were developed by ECL, and images were scanned. Representative blots out from four or five performed using at least three different experiments are shown (FIG. 11A). N, intact, naive rats (N1, N2, n=2); S, sham operated rats (S1, S2, n=12); I/R, 30-min ischemia followed by 30-min reperfusion rats (I/R1, I/R2, n=2); and A1, A2, A3, chronic alcoholic rats (n=3). Representative blot out from three performed is shown for LPS/D-Gal treatment using three rats for each time point; the I/R sample (30/30) was included for comparison (FIG. 11B).

FIG. 12A-12D are scans of blots (12A and 12B) and graphs (12C and 12D) showing time-dependent accumulation of the blood ASS and EST-1 after I/R in rats. Blood was withdrawn from rat heart following 30-min ischemia followed by different times of reperfusion as described in the Materials and methods. Proteins were separated by SDS-PAGE/western blot with antibody against ASS (FIG. 12A) and EST-1 (FIG. 12B). Images were captured and protein bands were calculated using ImageJ software (FIG. 12C, 12D). Representative blots from five performed using at least four different experiments are shown. N, intact, naive rats (n=5); S, sham operated rats (n=4); I/R, 30-min ischemia followed by 10-180 min of reperfusion (n=4).

FIG. 13 is a plot showing resolved naive brain lysate using the standard sized columns for CAX chromatography.

FIG. 14 is a plot showing the same brain fraction (see, FIG. 13) which was resolved by CAX chromatography using the newly tested (longer, smaller bore, smaller particle size) columns in a tandem configuration (cation exchange followed by anion exchange). Higher efficiency translates into greater resolution, with reduced fraction volume for easier transfer to 1D PAGE. This enhanced CAX chromatography is used to collect between 48 and 96 fractions into a 96-well filtration plate, for either direct digestion and loading onto RPLC-MSMS, or for further resolution by 1D-PAGE.

FIG. 15 are scans of gels showing a comparison of the columns where first 11 fractions are resolved by 1D polyacrylamide gel electrophoresis (1D-PAGE). Higher efficiency translates into greater resolution, with reduced fraction volume for easier transfer to 1D PAGE.

### DETAILED DESCRIPTION

A system and methods for resolution, identification and quantitation of complex biological mixtures are provided. In particular, the system comprises combined cationic and anionic exchange in tandem with gel electrophoresis to enable the rapid and efficient identification of proteins and/or peptides such as biomarkers indicative of a disease state. Furthermore, the invention provides protein visualization techniques that enable rapid identification of differential expression, or presence of, certain proteins in a biological sample relating to a certain biological or medical condition.

Definitions

Prior to setting forth the invention, it may be helpful to an understanding thereof to set forth definitions of certain terms that will be used hereinafter.

The term “capillary” as used in reference to the electrophoretic device in which electrophoresis is carried out in the methods of the invention is used for the sake of convenience. The term should not be construed to limit the particular shape of the cavity or device in which electrophoresis is conducted. In particular, the cavity need not be cylindrical in shape. The term “capillary” as used herein with regard to any electrophoretic method includes other shapes wherein the internal dimensions between at least one set of opposing faces are approximately 2 to 1000 microns, and more typically 25 to 250 microns. An example of a non-tubular arrangement that can be used in certain methods of the invention is the a Hele-Shaw flow cell. Further, the capillary need not be linear; in some instances, the capillary is wound into a spiral configuration, for example.

As used herein, the term “ion exchange efficiency” means the efficiency with which ions in a solution are exchanged with those bound to an ion exchange material. For example, ion exchange efficiency can be defined as E/M, where E is the percent of ions in a solution that are exchanged with the ions bound to an ion exchange resin, and M is the mass of the ion exchange resin. Ion exchange efficiency can be determined by, for example, passing equal volumes of water containing equal ion concentrations through the ion exchange media being measured, and then measuring how many of the ions have been exchanged. Ion exchange can easily be determined by methods known to those skilled in the art including, but not limited to, ultraviolet and visible absorption measurements, atomic absorption spectra, and titration. Therefore, the plurality of ion-exchange media used in the invention are easily determined based on desired ion exchange efficiencies. Ion exchange media are available through commercial sources.

“Marker” or “biomarker” in the context of the present invention refers to a polypeptide (of a particular apparent molecular weight) which is differentially present in a sample taken from patients having a disease, such as cancer, injury such as neural injury and/or neuronal disorders as compared to a comparable sample taken from control subjects (e.g., a person with a negative diagnosis, normal or healthy subject).

The phrase “differentially present” refers to differences in the quantity and/or the frequency of a protein and/or peptides present in a sample taken from patients having for example, neural injury as compared to a control subject. For example, a marker can be a polypeptide which is present at an elevated level or at a decreased level in samples of patients with neural injury compared to samples of control subjects. Alternatively, a marker can be a polypeptide which is detected at a higher frequency or at a lower frequency in samples of patients compared to samples of control subjects. A marker can be differentially present in terms of quantity, frequency or both.

A polypeptide is differentially present between the two samples if the amount of the polypeptide in one sample is statistically significantly different from the amount of the polypeptide in the other sample. For example, a polypeptide is differentially present between the two samples if it is present at least about 120%, at least about 130%, at least about 150%, at least about 180%, at least about 200%, at least about 300%, at least about 500%, at least about 700%, at least about 900%, or at least about 1000% greater than it is present in the other sample, or if it is detectable in one sample and not detectable in the other.

Alternatively or additionally, a polypeptide is differentially present between the two sets of samples if the frequency of detecting the polypeptide in samples of patients' is statistically significantly higher or lower than in the control samples. For example, a polypeptide is differentially present between the two sets of samples if it is detected at least about 120%, at least about 130%, at least about 150%, at least about 180%, at least about 200%, at least about 300%, at least about 500%, at least about 700%, at least about 900%, or at least about 1000% more frequently or less frequently observed in one set of samples than the other set of samples.

“Diagnostic” means identifying the presence or nature of a pathologic condition. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay, are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

A “crude biological sample” as used herein is any sample, for example, tissue, cell etc which is not subjected to any type of treatment but refers to for example, a homogenized tissue sample, a lysed cell and the like.

“Peak Capacity (n_{c})” is the number of peaks that can fit into a chromatogram.

“Fraction Volume (Vf)” is the volume required to collect an average peak.

Anion exchangers can be classified as either weak or strong. As used herein, a weak anion exchange medium” or “weak cationic exchanger” is one where the charge group is a weak base, which becomes deprotonated and, therefore, loses its charge at high pH. DEAE-cellulose is an example of a weak anion exchanger, where the amino group can be positively charged below pH˜9 and gradually loses its charge at higher pH values. A “strong anion exchanger” on the other hand, contains a strong base, which remains positively charged throughout the pH range normally used for ion exchange chromatography (pH 1-14).

Cation exchangers can also be classified as either weak or strong. A “strong cation exchange medium” or “strong cation exchanger” contains a strong acid (such as a sulfopropyl group) that remains charged from pH 1-14; whereas a “weak cation exchange medium” or “weak cationic exchanger” contains a weak acid (such as a carboxymethyl group), which gradually loses its charge as the pH decreases below 4 or 5.

The charge on the protein affects its behavior in ion exchange chromatography. Proteins contain many ionizable groups on the side chains of their amino acids as well as their amino- and carboxyl-termini. These include basic groups on the side chains of lysine, arginine and histidine and acidic groups on the side chains or glutamate, aspartate, cysteine and tyrosine. The pH of the solution, the pK of the side chain and the side chain's environment influence the charge on each side chain. The relationship between pH, pK and charge for individual amino acids can be described by the Henderson-Hasselbalch equation:

In general terms, as the pH of a solution increases, deprotonation of the acidic and basic groups on proteins occur, so that carboxyl groups are converted to carboxylate anions (R—COOH to R—COO—) and ammonium groups are converted to amino groups (R—NH³⁺ to R—NH₂). In proteins the isoelectric point (pI) is defined as the pH at which a protein has no net charge. When the pH>pI, a protein has a net negative charge and when the pH<pI, a protein has a net positive charge. The pI varies for different proteins.

Isolation and Quantitation

The methods of the present invention utilize a combination of methods conducted in series to resolve mixtures of proteins. The methods are said to be conducted in series because the sample(s) isolated in each method are from solutions or fractions containing proteins isolated in the preceding method, with the exception of the sample electrophoresed in the initial method. As used herein, the terms protein, peptide and polypeptide are used interchangeably and refer to a polymer of amino acid residues. The term also applies to amino acid polymers in which one or more amino acids are chemical analogues of corresponding naturally-occurring amino acids, including amino acids which are modified by post-translational processes (e.g., glycosylation and phosphorylation).

In a preferred embodiment, the present invention relates to a system and methodology for identifying protein patterns associated with predetermined biological characteristics. Another aspect relates to a system and methodology for identifying protein patterns associated with predetermined clinical parameters. A further aspect relates to a system and methodology for identifying protein patterns associated with predetermined medical conditions. Still, a further aspect relates to a system and methodology for identifying protein patterns associated with predetermined diseases.

In another preferred embodiment, the present invention also relates to a system and methodology for predicting the existence or non-existence of at least one predetermined biological characteristic. The present invention also relates to a system and methodology for predicting the presence of disease in an animal body, such as a mammal.

In other preferred embodiments, a system and methodology for rapidly identifying proteins associated with disease or other biological conditions are used as biomarkers in diagnostic applications. The present invention also relates to a system and methodology for using biomarker proteins as a therapeutic target for treatment of disease or other biological conditions. The present invention also relates to a system and methodology for discovering proteins that are useful as imaging or therapeutic targets of disease.

In another preferred embodiment, protein biomarkers are identified for monitoring the course of a disease, and for determining appropriate therapeutic intervention. Additional features of the invention will be set forth in part in the description which follows, and in part will become apparent to those having ordinary skill in the art upon examination of the following or may be learned from practice of the invention.

As a non-limiting example intended for illustration purposes, enhanced tandem cationic/anionic ion-exchange chromatography increased protein retention to 88% for uniform protein distribution across 25 or more fractions per sample. Paired fractions from each sample were loaded on conventional 1D-PAGE for differential comparison with a loading reproducibility of 94% while avoiding gel-to-gel variability issues. The CAX-PAGE theoretical peak capacity of 3570, extendable to 7600, was on par with other 2D protein separations; however resolving power is further extended using subsequence peptide separations. From a differential target list based on a two fold band intensity difference between samples, matched bands were in-gel digested and separated by capillary reverse phase liquid chromatography inline with a quadruple ion trap tandem mass spectrometer. The 4D theoretical peak capacity is about 1.43×10⁸, with a saturation factor of only 3.5% assuming a peptidome of 5×10⁵ proteins fragmented into 100 unique peptides. Differential analysis by CAX-PAGE/RPLC-MSMS is effectively demonstrated using a neuroproteomic model comparing cerebellum and cortex rat tissues. Protein separations revealed 137 distinct differential targets, 67% more than with alternative 2D-DIGE technology, of which 33 were randomly selected for subsequent peptide analysis. Verifiable protein identification was determined in 85% of cases, out of which 89% had semi-quantitative peptide data validating differential CAX-PAGE band intensity determinations. Further, matching gel band and identified protein masses from 16 to 273 kDa corroborated protein determination and demonstrated the platform's effective mass range.

According to one embodiment, the invention combines cationic and anionic exchange separation, herein termed “CAX chromatography”, with the resolving and visualization power of 1D-PAGE. Complex protein mixtures, nearly all biological samples, can be resolved by ionic-strength then mass by this multi-dimensional separation technique. Low salt and surfactant concentrations can be tolerated, while samples with high salt or surfactants can be pre-cleaned by dialysis or precipitation procedures. The sample is then injected onto tandem cationic and anionic columns or a mixed bed column (containing both media). The low percentage of neutral proteins run through the column(s) and are collected in the first few fractions. The retained proteins are eluted by increasing the counter-ion salt concentration in a gradient fashion with fraction collection. For example, the gradient is a matter of proportioning two or more mobile phases together. As an illustrative example, 20 mM Tris Buffered water and B: 1 M salt (NaCl) in 20 mM Tris Buffered water are mixed proportionally from 0% to 100% B in multiple linear gradients—performed through computer control of two pumps pushing at different rates (one for each mobile phase). The strategy with CAX optimization is that gradient segments can be added (e.g., 1-20 segments) each with a different rate of mixing (i.e., gradient slope) to allow even separation of proteins across the fractions collected. Different sample types (e.g. tissue vs. biofluids) requires such optimization. Samples can also be collected at different flow rates (0.010 mL/min to 2.5 mL/min) based mainly on column size (2 mm to 2 cm i.d.). For example, as few as 9 to as many as 50 fractions per sample can be collected, with volumes from 100 μl to 2 ml.

In accordance with the invention, a gradient step is as small as 2.5% change in A:B to as much as 50% change in as little as 1 min to as long as 60 min. Therefore, gradient steps can be tailored to the sample at hand.

Any type of ion-exchange material can be used. For example, ion exchange resins can be cationic, anionic, mixtures of cation and anionic, or biologically related. Examples of ion exchange resins useful in this invention include, but are not limited to, those made of cross-linked polyvinylpyrolodone and polystyrene, and those having ion exchange functional groups such as, but not limited to, halogen ions, sulfonic acid, carboxylic acid, iminodiacetic acid, and tertiary and quaternary amines. Specific examples of cationic ion exchange resins include, but are not limited to: AMBERJET™ 1200(H); Amberlite™ CG-50, IR-120(plus), IR-120(plus) sodium form, IRC-50, IRC-50S, and IRC-718; Amberlyst™ 15, 15(wet), 36(wet), A-21, A-26 borohydride, bromide, chromic acid, fluoride, and tribromide; and DOWEX™ 50WX2-100, 50WX2-200, 50WX2-400, 50WX4-50, 50WX4-100, 50WX4-200, 50WX4-200R, 50WX4-400, HCR-W2, 50WX8-100, 50WX8-200, 50WX8-400, 650C, MARATHON™ C, DR-2030, HCR-S, MSC-1, 88, CCR-3, MR-3, MR-3C, and Retardion™. Specific examples of anionic ion exchange resins include, but are not limited to: AMBERJET™ 4200(CI); Amberlite™ IRA-67, IRA-400, IRA-400(CI), IRA-410, IRA-743, IRA-900, IRP-64, IRP-69, XAD-4, XAD-7, and XAD-16; AMBERSORB™ 348F, 563, 572 and 575; DOWEX™ 1X2-100, 1X2-200, 1X2-400, 1X4-50, 1X4-100, 1X4-200, 1X4-400, 1X8-50, 1X8-100, 1X8-200, 1X8-400, 21K Cl, 2X8-100, 2X8-200, 2X8-400, 22 Cl, MARATHON™ A, MARATHON™ A2, MSA-1, MSA-2, 550A, 66, MARATHON™ WBA, and MARATHON™ WGR-2; and Merrifield's peptide resins. A specific example of mixed cationic and anionic resins is Amberlite™ MB-3A. Specific examples of biologically related resins that can be used in the processes and products of the invention include, but are not limited to, Sephadex™ CM C-25, CM C-50, DEAE A-25, DEAE A-50, QAE A-25, QAE A-50, SP C-25, and SP C-50. These cationic, anionic, mixed cationic and anionic, and biologically related ion exchange resins are commercially available from, for example, Aldrich Chemical Co., Milwaukee, Wis., or from Rohm and Haas, Riverside, N.J. Additional examples of ion exchange resins include, but are not limited to AG-50W-X12, Bio-ReX™ 70, and Chelex™ 100, all of which are tradenames of Bio-Rad, Hercules, Calif.

Examples of functional groups used in ion exchange chromatography for selection of weak vs. strong anionic or cationic media are as follows:

| Functional Group | pK Value | Characteristic | Description |
|---|---|---|---|
| TMAE-Group | pK > 13 | strongly basic | Trimethylammonium- |
| | | | ethyl- |
| DEAE-Group | pK 11 | weakly basic | Diethylaminoethyl- |
| DMAE-Group | pK 8-9 | weakly basic | Dimethylaminoethyl- |
| COO-Group | pK 4.5 | weakly acidic | Carboxy- |
| SO3-Group | pK < 1 | strongly acidic | Sulfoisobutyl- |
| SE-Group | pK < 1 | strongly acidic | Sulfoethyl- |

A variety of buffers at different pH values (e.g., Tris-HCL, HEPES, and multi-pH phosphate buffers) can be used to tailor charge distribution. Additionally, a pH gradient can be used in place of the salt gradient mentioned here—this would be more akin to isoelectric focusing used in 2D-PAGE. The benefit of a salt gradient is that all proteins can be maintained at the same pH, preferably neutral, to prevent denaturing. Fractions are then concentrated down with micro-spin tubes, to which gel electrophoresis sample buffer is added for reconstitution and collected for direct loading onto one-dimensional polyacrylamide gel electrophoresis (1D-PAGE). The gels are then visualized with traditional protocols. A variety of conventional staining techniques, such as but not limited to, Coomassie stain for detection of high-concentration proteins, or more sensitive stains (e.g., silver or Sypro ruby) for detection of less abundant proteins, may be used in accord with the principles of the invention. This method is referred to herein as CAX/1D-PAGE.

In another preferred embodiment, the CAX/1D-PAGE system is used for differential comparison of complex biological mixtures. Two strategies were performed to demonstrate differential proteomic analysis. The first utilized the reproducibility of CAX/1D-PAGE to run two different samples sequentially (e.g., a control and a treated sample) by CAX chromatography and then load paired fractions side-by-side on 1D-PAGE. Though differences can be observed by close examination of adjacent lanes, a visualization method was developed to observe expression differences by a positive (green) or negative (red) color shift from equal expression (yellow) to take advantage of the human eye's keen ability to detect color. A second differential expression strategy utilizes cyanine dye technology in a similar fashion to that applied with 2D-PAGE. This embodiment has the advantage of being more reproducible as both control and treated samples can be mixed and run through CAX/1D-PAGE separation together since they can be visualized via different fluorescence conditions. However, cyanine dyes are less sensitive and more difficult to use than other stains such as silver or sypro ruby for visualization of less concentrated proteins. Both strategies are useful depending on the experiment. Those skilled in the art will appreciate that various dyes may be implemented in accordance with the teachings herein.

In accordance with the invention, linear gels can be from about 4% acrylamide to about 18% acrylamide (e.g. 4, 5, 6, 7.5, 8, 10, 12, 12.5, 14, 15, 16, 18%). Gradient gels can be in differing gradients such as for example: 4-15%, 4-20%, 8-16%, 10.5-14%, 10-20%. Any size gel can be used, for example commercially available gels are about 20 cm SDS-PAGE with differing numbers of gel lanes (10 to 26 wells) and gel thicknesses (1 mm to 1.5 mm).

In another embodiment, CAX is implemented in combination with second dimensional liquid chromatography for separation of proteins and peptides. As discussed supra, ion-exchange chromatography has been used as a first stage to multi-dimensional chromatography. In both cases, sample fractionation can be enhanced by employing CAX chromatography in place of either cationic (SCX) or anionic (SAX) ion-exchange chromatography alone. This is based on the same principle as illustrated in the Examples which follow, that all acidic and basic molecules will bind with CAX. This embodiment shows superior, unexpected results when conducting online 2D-LC separations for performing shotgun proteomics or for analysis of post-translationally modified (PTM) proteins, particularly for those proteins/peptides that are modified with highly charged groups (e.g., phosphate). Such PTMs can be further elucidated with special stains (that are selective to PTMs of interest. The present invention permits high-resolution separation of complex protein mixtures, particularly biological samples derived from tissue, body fluids, and all forms of cell lysates, with visualization using conventional gel stains.

In another preferred embodiment, differential proteome analysis of complex protein mixtures—the comparison of protein expression between two samples (e.g., biomarker discovery, sub-proteome analysis, etc.) is conducted using the methods of the invention. This technology is also used to visualize post-translationally modified subproteomes by use of special selective gel stains.

In another preferred embodiment, the CAX chromatography is placed online with additional liquid chromatography (e.g., reverse phase, size exclusion, etc.) to provide increased sample fractionation and two dimensional resolution. This can be applied to both protein and peptide mixtures, including those with post-translationally modified components with separation at the preparatory down to the capillary scale. The CAX chromatography is used to pre-fractionate a complex mixture for multiple 1D-PAGE and/or 2D-PAGE analysis.

Accordingly, in one embodiment, the subject invention pertains to a method of identifying at least one biomarker comprising obtaining a biological sample from a patient known to have an injury, disorder or pathological condition (test sample(s)); obtaining at least one biological sample from a patient known not to have such injury or pathological condition (control sample(s)); sequentially performing CAX chromatography to said biological samples to produce fraction samples; subjecting fraction samples to electrophoresis in a gel; visualizing proteins in said gel; identifying presence of proteins in one sample not present in another sample, wherein differential presence indicates a biomarker candidate. Preferably, subjecting fraction samples to electrophoresis comprises performing 1-D PAGE. Also preferred is running electrophoresis with fractions from the test sample side-by-side with corresponding fractions from the control sample. Visualizing the proteins comprises staining fractions from the control sample with a first dye and staining fractions from the test sample with a different dye. The corresponding fraction samples may be overlaid whereby different colors generated indicate the presence of a protein in one or the other sample, or both. (See, the Examples which follow). The method of identifying biomarkers can be applied to identify biomarkers relating to, but not limited to neurological injuries, disorders and diseases; cancer; autoimmune disorders; stress; exposure to toxins; and joint disease. In the case of identifying biomarkers for brain injury, blood, serum or central spinal fluid samples from individuals known to have a brain injury are compared to individuals known not to have a brain injury. The sample may be tissue homogenate, urine, blood, CSF, serum or other biological fluid present in the body.

The markers identified by the methods taught herein may be used to diagnosed multiple medical conditions, including but not limited to, brain injuries, such as those caused by accidental trauma, strokes, etc.; presence of tumors; autoimmune diseases; and neurodegenerative diseases. Furthermore, the CAX chromatography methods may be used as research techniques for basic research. U.S. Patent applications 20040066955; 20030232396; and 20030211531; and PCT publication WO 2002/US0019813 discuss methods of identifying biomarkers.

In another preferred embodiment, a method of isolating and differential quantitative analysis of proteins and/or peptides in complex biological mixtures, said method comprising: obtaining a crude biological sample; subjecting the sample to a bi-phasic ion-exchange chromatography and obtaining fractions; running the fractions obtained in order of elution side-by-side on a polyacrylamide gel electrophoresis allowing for differential comparison; quantifying bands obtained by polyacrylamide gel electrophoresis by densitometric scanning; selecting bands which are differentially expressed at least about two-fold as compared to a normal control; digesting the differentially expressed bands with enzyme; subjecting the enzyme digested bands to capillary reverse phase liquid chromatography online in tandem with mass spectrometry; thereby, isolating and quantifying the isolated proteins and/or peptides. Preferably, quantification of isolated proteins is validated by comparing the protein amounts with gel band density.

Accordingly, the bi-phasic ion ion-exchange chromatography comprises at least a plurality of gradients, preferably, the bi-phasic ion exchange chromatography comprises at least a two step gradient, preferably, the bi-phasic ion exchange chromatography comprises a three step gradient, preferably, the bi-phasic ion exchange chromatography comprises a five step gradient, preferably, bi-phasic ion exchange chromatography comprises a ten step gradient, preferably, the bi-phasic ion exchange chromatography comprises between about a two step gradient up to a twenty step gradient. In accordance with the invention, the gradient is optimized depending on the viscosity of the mixture, the complexity of the biological sample and the like and can include a plurality of gradients.

In another preferred embodiment, the polyacrylamide gel comprises a gradient of between about 1% up to 50% and/or can be a gel without a gradient. The percentage of the gel can be from about 1% to about 50% and the gradient can be changed to isolate bands of close molecular weights during the differential analysis.

In accordance with the invention, the bands on the gel can be visualized using any number of dyes. For example, Coomassie blue, silver staining, Sypro Ruby, cyanine dyes and the like.

In another preferred embodiment, bands on the gel digested by enzymes selected from the group consisting of hydrolases, esterases, carbohydrases, nucleases, deaminases, amidases, proteases, hydrases, fumarase, enolase, aconitase carbonic anhydrase, oxidases, dehydrogenases; transglycosidases; transphosphorylases phosphomutases, transaminases; transmethylases, transacetylases, desmolases, isomerases; and ligases. Preferably, the enzyme is a tryptase.

In another preferred embodiment, the enzyme digested bands are subjected to reverse phase liquid chromatography. Preferably, the n_{c} values of the reverse phase liquid chromatography are between about 100 to about 250.

In another preferred embodiment, the fractions eluted from the reverse phase liquid chromatography directly flow into the mass spectrometry and separated by mass-to-charge. Preferably, the n_{c} values are at least about 1×10⁵, preferably, the n_{c} values are about 1×10⁶, preferably, the n_{c} values are about 1×10⁷, preferably, the n_{c} values are about 1×10⁸, preferably, the n_{c} values are about 1×10⁹, preferably, the n_{c} values are about 1×10¹⁰.

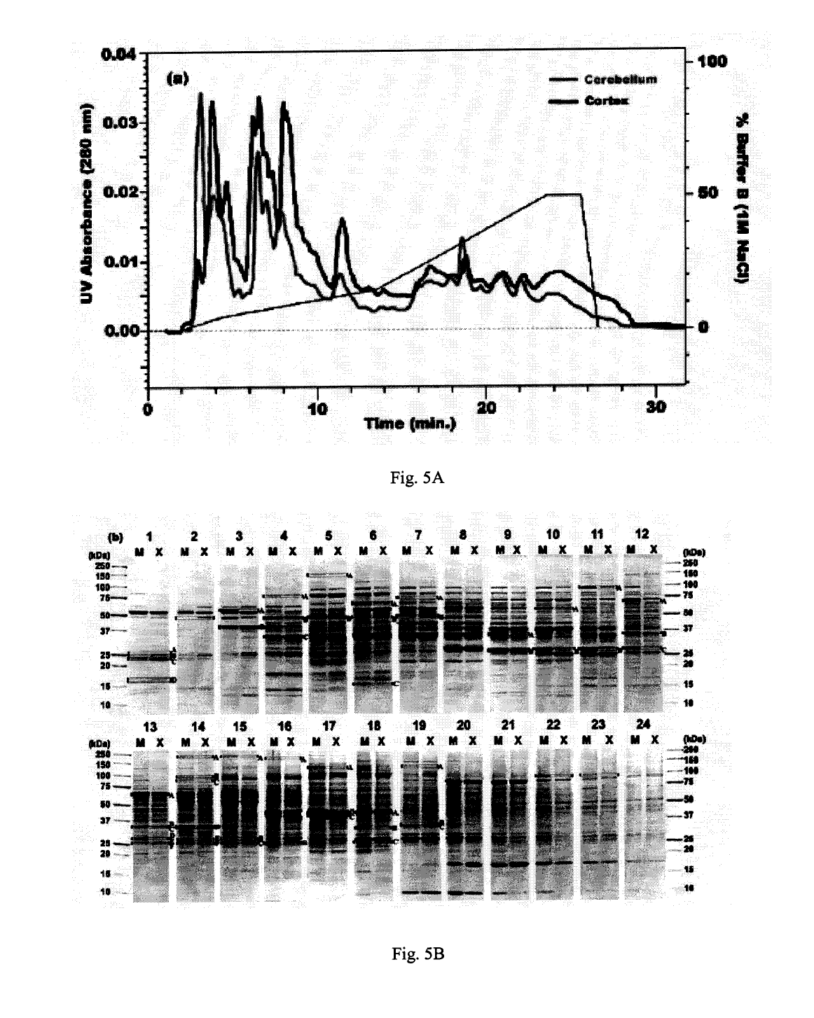

An illustrative example, without limiting the invention in any way, of differential analysis is as follows: The potential of CAX-PAGE is realized with its ability to provide differential expression maps for subsequent targeted differential RPLC-MSMS analysis. As a test case, proteomic differences between cerebellum and cortex regions of rat brain were explored. It was expected that the compliment of proteins would be similar in both tissues, but that expression would differ. Clear chromatographic differences in FIG. 5a are observed between cerebellum and cortex lysates sequentially separated by CAX. For differential analysis, fractions from each run are paired and run side-by-side on 1D-PAGE (FIG. 5b), whereby problems of gel-to-gel reproducibility are avoided by always comparing matching fractions on the same gel. Side-by-side fraction pairing as in FIG. 5b allows for direct visualization of differential expression using simple, cost efficient, visible stains (e.g., Coomassie Blue, silver, Deep Purple). Fluorescent stains such as Sypro Ruby also work well, though they require a more expensive fluorescence scanner (three times the cost for the liquid chromatography station). Whether visible or fluorescent stains are used, images are easily assessed with the Phoretix 1D software. Automatic processing is performed to identify gel lanes, providing a clear boundary along the x-axis. Band height is also distinguishable, though fainter bands tend to require manual verification. Band intensity is automatically calculated along with band mass based on calibration with a traditional protein marker. Data is then output to an excel spreadsheet with adjacent bands lined up between lanes. A threshold set at 100% difference in band intensity is applied to generate a list of target bands for further analysis, thereby minimizing mass spectrometry workload in comparison with shotgun proteomic protocols.

Samples

The methods of the invention can be used with a wide range of sample types. Essentially any protein-containing sample can be utilized with the methods described herein. The samples can contain a relatively small number of proteins or can contain a large number of proteins, such as all the proteins expressed within a cell or tissue sample, for example.

In preferred embodiments, tissue and cell culture samples are clarified of large cellular debris—clumps of cell parts that are not easily broken up. This can be done by any method such as described in the Examples which follow. For example, centrifugation and running the supernatant through a 0.1 μm centrifugal filter. Generally, nothing else need be done (no need to remove salts, or mix in other compounds), though highly viscous liquids may require dilution prior to loading e.g. tissue lysate, cell culture lysate, and CSF.

Samples can be obtained from any organism or can be mixtures of synthetically prepared proteins or combinations thereof. Thus, suitable samples. can be obtained, for example, from microorganisms (e.g., viruses, bacteria and fungi), animals (e.g., cows, pigs, horses, sheep, dogs and cats), hominoids (e.g., humans, chimpanzees, and monkeys) and plants. The term “subject” as used to define the source of a sample includes all of the foregoing sources, for example. The term “patient” refers to both human and veterinary subjects. The samples can come from tissues or tissue homogenates or fluids of an organism and cells or cell cultures. Thus, for example, samples can be obtained from whole blood, serum, semen, saliva, tears, urine, fecal material, sweat, buccal, skin, spinal fluid, tissue biopsy or necropsy and hair. Samples can also be derived from ex vivo cell cultures, including the growth medium, recombinant cells and cell components. In comparative studies to identify potential drug or drug targets, one sample can be obtained from diseased cells and another sample from non-diseased cells, for example.

Variations of Analysis

The methods of the invention use any variety of analyses for quantitation. For example, densitometric analysis, infra-red spectroscopy, nuclear magnetic resonance spectroscopy, UV/VIS spectroscopy and complete or partial sequencing. Coupling the electrophoresis to any mass spectroscopy (MS) methods is within the scope of the invention. A variety of mass spectral techniques can be utilized including several MS/MS methods and Electrospray-Time of Flight MS methods. Such methods can be used to determine at least a partial sequence for proteins resolved by the methods such as a protein sequence tag.

Advantages

As mentioned above, CAX chromatography can be used in conjunction with 2D-PAGE analysis. CAX chromatography/1-D PAGE has certain advantages over the use of 2D-PAGE alone. For example, the CAX gradient can be optimized to provide even fractionation of proteins, or to emphasize a particular area. The number of fractions and associated gel lanes is limited only by the amount of time needed to process the samples—this means that resolution can be expanded indefinitely to provide significantly higher separation of proteins over the limited format size of 2D-PAGE. Many 1D-PAGE acrylamide compositions can be used to emphasize resolution at higher, lower, or intermediate protein mass. 2D-PAGE is limited in format. All acidic and basic proteins are separated by CAX prior to second dimensional separation, as compared to the limited p1 range of isoelectric focusing strips used for 2D-PAGE. The increased resolving power of CAX/1D-PAGE provides easier visualization of low and high concentration proteins—greater dynamic range based on increased resolving capability. Hydrophobic proteins are retained and separated by CAX/1D-PAGE. Components such as Urea or Chaps do not need to be added to perform CAX. Reproducibility is improved as only side-by-side lanes need be compared by 1D-PAGE and distortion is primarily in one direction. The use of a salt gradient over a pH gradient allows proteins to be kept in their native state.

The following examples are offered by way of illustration, not by way of limitation. While specific examples have been provided, the above description is illustrative and not restrictive. Any one or more of the features of the previously described embodiments can be combined in any manner with one or more features of any other embodiments in the present invention. Furthermore, many variations of the invention will become apparent to those skilled in the art upon review of the specification. All publications and patent documents cited in this application are incorporated by reference for all purposes to the same extent as if each individual publication or patent document were so individually denoted. By their citation of various references in this document, Applicants do not admit any particular reference is “prior art” to their invention.

### EXAMPLES

Materials and Methods

Sample Preparation.

Male Sprague Dawley rats (five) purchased from Harlan (Indianapolis, Ind.) were acclimated for 7 days prior to sacrificing. The rats were then anesthetized with 4% isoflurane in a carrier gas of 1:1 O₂/N₂O (4 minutes), and were perfused with 0.9% saline transcardially prior to decapitation via guillotine. Cerebellum and cortex brain regions were dissected and transferred to microfuge tubes kept on dry ice. Sections were snap frozen in liquid nitrogen then ground to a fine powder via mortar and pestle kept on dry ice. Powder was scrapped into chilled microfuge tubes to which 0.1% SDS lysis buffer (300 μl) was added containing 150 mM NaCl, 3 mM EDTA, 2 mM EGTA, 1% IGEPAL (all from Sigma-Aldrich, St. Louis, Mo.), one tablet of Complete Mini Protease Inhibitor Cocktail (Roche Diagnostics, Mannheim, Germany) and 1 mM sodium vanadate (Fisher Scientific, Fair Lawn, N.J.) with the sample solution brought to neutral pH using Tris-base (Sigma-Aldrich). Cell lysis was conducted over 3 hours at 4° C. with hourly vortexing. Lysates were spun down at 14,000 rpm at 4° C. for 10 minutes to remove DNA, lipids, and particulates. Supernatants were then filtered through 0.1 μm Millipore Ultrafree-MC filters (Bedford, Mass.) for further clarification. Protein concentrations were determined via Bio-Rad DC Protein Assay (Hercules, Calif.), after which pooled (n=5) 1 mg cortex and 1 mg cerebellum samples were prepared for differential comparison.

Ion-Exchange Chromatography.

A Bio-Rad (Hercules, Calif.) Biologic DuoFlow system with QuadTec UV detector and BioFrac fraction collector was used with Uno series SAX (Q1) and SCX (S1) pre-packed ion exchange columns. For CAX chromatography, S1 and Q1 columns were placed in series. Buffers consisted of ice cold 20 mM Tris-HCl (pH 7.5 molecular biology grade, Fisher Scientific) in HPLC water (Burdick & Jackson, Muskegon, Mich.)(mobile phase A). A two step elution gradient was performed with 1 M NaCl (Fisher Scientific, crystalline 99.8% certified) (mobile phase B) at a flow rate of 1 mL/min with a linear transition from 0 to 15% B in 12.5 mL followed by 15 to 50% B in 7 mL. The composition was then held at 50% B for 2 mL, and then re-equilibrated to 0% B in 1 mL. An optimized three step gradient was used for differential analysis. At a flow rate of 1 mL/min, the first linear transition was from 0 to 5% B in 2.5 mL then from 5% to 15% in 9 mL followed by 15% to 50% in 10 mL. Again the composition was held at 50% B for 2 mL, and re-equilibrated to 0% B in 1 mL. UV chromatograms were collected at a wavelength of 280 nm. Twenty-five 1 mL fractions were autonomously collected via the BioFrac fraction collector into 1.5 mL screw cap microfuge tubes (RPI, Mt. Prospect, Ill.) kept on ice.

1D-SDS-PAGE.

Fractions collected during ion exchange chromatography were concentrated via Millipore YM-30 centrifugal filters. Each fraction was spun through filters pre-washed with 500 μl of HPLC water as two 500 μl sequential portions at 13,500 rpm for 20 minutes. Laemmli sample buffer (Bio-Rad, with 5% BME) was added to the retentate, and incubated for 10 minutes prior to collection by centrifugation at 3,500 rpm for 3 minutes. The supernatant for each fraction was boiled at 85° C. for two minutes, and then loaded onto Invitrogen Novex 10-20% gradient 1 mm 10 well gels in a Tris-glycine buffer system (Carlsbad, Calif.) alongside a lane of Amersham Biosciences Rainbow Marker (Piscataway, N.J.) for initial studies. Differential analysis between cerebellum and cortex tissues was performed by pairing fractions for loading side-by-side (i.e., cerebellum fraction 1 next to cortex fraction 1, etc.) on Bio-Rad Criterion 10-20% gradient 1 mm 18 well gels in a Tris-glycine buffer system.

Protein Retention and Recovery of CAX

Protein recovery was evaluated with SAX, SCX, and combined phase CAX. A constant protein amount (750 μg) in a 100 μl injection of the previously described cerebellum lysate was loaded. A 1 mL/min isocratic flow was maintained for 9 minutes to allow unretained proteins to flow through. The mobile phase composition was then increased in 1 minute and held for 9 minutes at 50% B to elute bound proteins collected in a normal gradient run. This was followed by an additional increase to 100% B in 1 minute which was held for another 9 minute to check for additional protein. Throughout, UV absorbance was monitored at 280 nm and 1 mL fractions were collected, each concentrated using Millipore YM-30 centrifugal filters and analyzed via Bio-Rad DC Protein Assay.

CAX-PAGE Coomassie Blue Imaging.

Gels were visualized by regressive staining using concentrated Bio-Rad Coomassie Blue R250 for 20 minutes and destained in 40% HPLC grade ethanol (EM Science, Gibbstown, N.J.)/10% acetic acid (ACS Plus grade, Fisher) for approximately two hours. Images were captured with an Epson 1640 XL flatbed scanner (Long Beach, Calif.) and saved as 8-bit TIFF files. Differential analysis of Coomassie blue stained gelswas performed using Phoretix 1D (Nonlinear Dynamics, Newcastle, UK) gel image analysis software. Band intensities were automatically calculated and manually verified for bands above a preset threshold. Intensities were output to Excel (Microsoft, Redmond, Wash.) for differential evaluation. Manual confirmation was aided by superimposing cerebellum lanes false colored red over adjacent cortex lanes false colored green creating gradient color lanes for each fraction. Image contrast was improved by adjusting RGB color balance to emphasize mid tones over shadows.

2D-DIGE.

Cerebellum and cortex samples (1 mg each) were prepared from the same pooled material used for CAX-PAGE. Each was adjusted to 2% SDS, followed by TCA precipitation. The pellet was air dried and resuspended in 150 μl of pH 8.8 urea lysis buffer. Benzonase Nuclease (Novagen, Madison, Wis.) and 5 mM magnesium chloride (Fisher) were added, incubating the mixture for 30 minutes on ice to degrade nucleic acids. The solution was clarified by centrifugation with a Beckman Coulter (Fullerton, Calif.) Airfuge at 100,000 g for 30 minutes. The supernatant was dialyzed against the urea lysis buffer overnight at room temperature. A 50 μg portion of cortex and cerebellum lysate was labeled with Cy3 and Cy5 minimal dyes (Amersham Biosciences), respectively, using the manufacturer's suggested protocol. Cyanine labeled samples were combined with 275 μg both of unlabeled cortex and cerebellum lysates. The solution was adjusted to 0.2% of IPG pH 3 to 10 buffer (Amersham Biosciences) and 100 mM DTT with a trace of Orange G stain (Fisher). An 18 cm non-linear pH 3 to 10 IPG strip (Amersham Biosciences) was rehydrated in the mixed sample under oil overnight at room temperature. Proteins were focused on the strip at 8 kV until migration was complete (65 kVhrs). Proteins in the strip were reduced with 100 mM DTT in the reaction buffer 50 mM pH 6.8 Tris-HCl, 6 M Urea, 30% glycerol, and 2% SDS. Alkylation was performed with 2.5% iodoacetamide in the same reaction buffer. The strip was mounted atop a Bio-Rad precise 8-16% Tris glycine gel, and run for 6 hrs at 25 mA and 24° C. Separate Cy3 and Cy5 images were collected on an Amersham Typhoon 8600 fluorescence imager, and processed with Phoretix 2D software (Nonlinear Dynamics).

In-Gel Digestion.

Gels were thoroughly rinsed with HPLC water. Target differential bands were excised and dissected into four cubes and placed in 0.5 μl tubes. Each was washed with HPLC water, then 50% 100 mM ammonium bicarbonate (Fisher)/50% acetonitrile (Burdick-Jackson, HPLC grade). Pieces were dehydrated with 100% acetonitrile and dried by speedvac (ISS110, Thermo Savant, Milford, Mass.). Cubes were rehydrated with 50 μl of 10 mM dithiothreitol (Calbiochem, San Diego, Calif.) in 50 mM ammonium bicarbonate and incubated for 30 minutes at 56° C. Dithiothreitol was replaced by 50 μl of 55 mM iodoacetamide (Calbiochem) in 50 mM ammonium bicarbonate, and reacted for 30 minutes in the dark at room temperature. Gel pieces were washed with 50 mM ammonium bicarbonate, and dehydrated with 100% acetonitrile followed by speedvac. Rehydration was performed with 15 μl of a 12.5 ng/μl trypsin solution (Promega Gold, Madison, Wis.) for 30 minutes at 4° C., then 20 μl of 50 mM ammonium bicarbonate was added and left at 37° C. overnight for digestion. The supernatant along with two 50% acetonitrile/5% acetic acid extractions were placed into a new tube. The peptide extract was dried by speedvac and resuspended in 20 μl 4% acetonitrile/0.4% acetic acid.

Capillary RPLC-MSMS.

Capillary RPLC tandem ion trap mass spectrometry was employed for protein identification as described previously with some modifications. Nanoflow reverse phase chromatography was performed with a 100 μm i.d.×5 cm capillary column packed in-house with Agilent (Palo Alto, Calif.) 3 μm C-18 particles behind an Upchurch 0.5 μm PEEK microfilter assembly. The integrated polymerized frit was replaced with a pulled emitter made from 25 μm i.d. capillary affixed to the other end of the microfilter assembly. Thirty minute gradients, 4% HPLC acetonitrile/0.4% acetic acid (Fisher, Optima grade) to 60% acetonitrile/0.4% acetic acid, were used to elute tryptic peptides. Tandem mass spectra were collected on a ThermoElectron (San Jose, Calif.) LCQ Deca XP-Plus using data-dependant analysis. Collected data were searched against the trypsin indexed complete NCBI RefSeq mammalian database filtered for rat taxonomy using ThermoElectron Bioworks Browser (version 3.1). We report protein identifications made with two or more peptides matched with strict cross correlation values of Xc≧1.8, 2.5, and 3.5 for +1, +2, and +3 charge states, respectively. Data filtering was performed with DTASelect, and cerebellum vs. cortex MSMS data was compared using Contrast software.

### Example 1

### CAX Chromatography—First Dimension

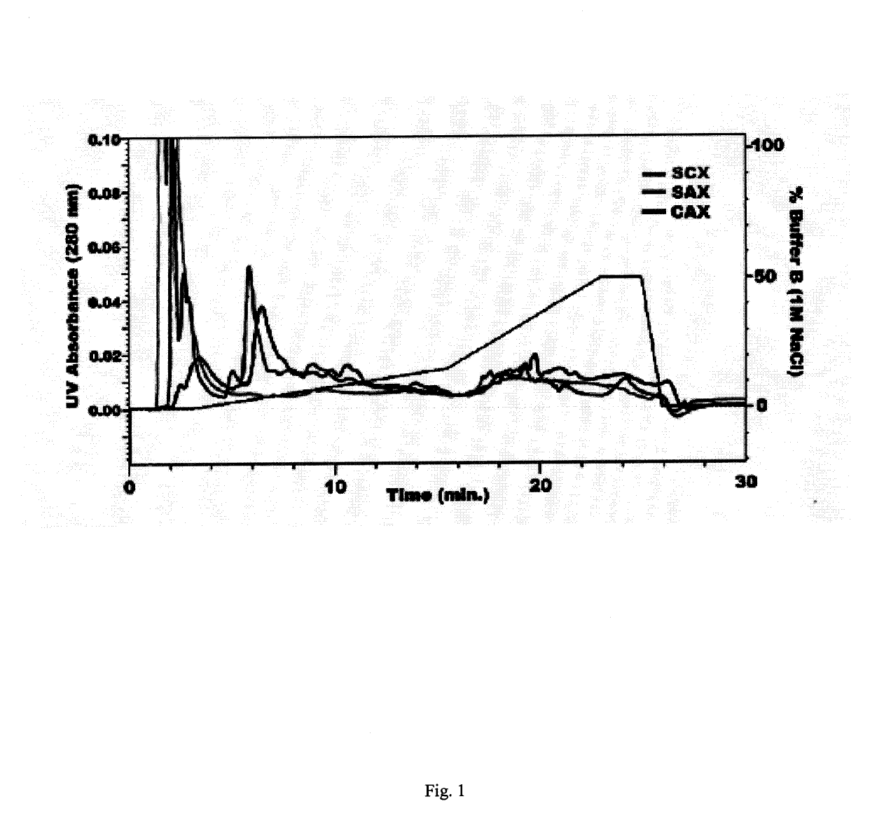

The majority of proteins in biological samples such as tissue lysates or body fluids retain regions of significant charge on their external surfaces when at physiological pH. Considered together, the net charge of these external regions approximately half of the time is negative and somewhat less than half of the time is net positive. Though in reality regions of external charge act independent of net charge, a general explanation for performing combined SCX and SAX is that categorically CAX will retain positively and negatively charged proteins rather than predominantly those of one net polarity. FIG. 1 illustrates the difference in gradient separation of a complex brain tissue lysate with independent SCX, SAX, and CAX chromatography. The single ion exchangers allow a significant portion of the proteome to flow through unretained, as evidenced by the large peak at the beginning of the chromatograms. CAX binds most proteins by charge interaction leaving generally net neutral proteins that despite regions of low charge density will not bind in the presence of background counter ions. In practice, a portion of this flow through fraction is resolved through hydrophobic interaction into approximately 4 flow-through fractions. FIG. 1 shows tandem cationic anionic ion-exchange (CAX) chromatography with superior protein retention. Strong-cationic ion-exchange (SCX), strong-anionic ion-exchange (SAX), and tandem cationic and anionic ion-exchange (CAX) chromatograms are shown overlaid for 1 mg of rat cerebellum brain lysate. Chromatograms are identically scaled at 280 nm; a more than 5 fold reduction in absorbance is observed at the start of the CAX chromatogram over the other two. Timing of the two-stage gradient is as indicated.

Common ion exchange salts, such as sodium or potassium chloride, provide both the cationic and anionic counter-ions necessary for CAX chromatography maintained in traditional low molarity buffers, such as Tris-HCl, HEPES, and variable pH phosphate buffers. Co-elution of both acidic and basic proteins by CAX chromatography is accomplished with a standard salt gradient where proteins elute off the column based on ionic strength. Initially a two stage gradient (0-15% B in 12 minutes, 15-50% B in 7 minutes) was optimized based on providing a uniform UV absorption across the entire chromatogram, presumably to provide even protein distribution across a targeted 25 fractions for maximal resolution. Further gradient optimization was required.

### Example 2

### Coupling to 1D-PAGE—Second Dimension

Orthogonal to ionic-strength, protein mass is used in the second dimension with 1D-PAGE to further resolve the complex brain lysate. A fraction volume of 1 mL, practical with the CAX flowrate and the BioFrac fraction collector, generally encompassed elution of entire proteins with CAX half-height peak widths generally in the order of 0.25 mL. A foreseen difficulty of CAX common with other fractionation strategies is that proteins can break across two fractions, fortunately this statistically is less likely at lower concentration when otherwise the problem would be most dramatic. Another complication is that the fraction volume is large relative to the loading volume of a gel. Microtube centrifugal filters were used to concentrate fractions to a manageable volume. A mass cutoff of 30 kDa was selected based on its association with relative pore size and not mass. Proteins>5 kDa are routinely retained with this filter, while the larger pore size provides more rapid processing than similar 10 or 3 kDa filters. To meet the gel loading volume requirements, retentate was brought close to dryness to allow the addition of 20 μl of 2× Laemmli sample buffer, which more effectively resolubilize protein than Tris-HCl buffer, while maintaining a small volume (reproducibly 35 μl±5 μl). On occasion, a random fraction would take longer to run through the filter, potentially due to manufacturing variability in the membrane pore size; though no obvious effect on protein retention was observed.

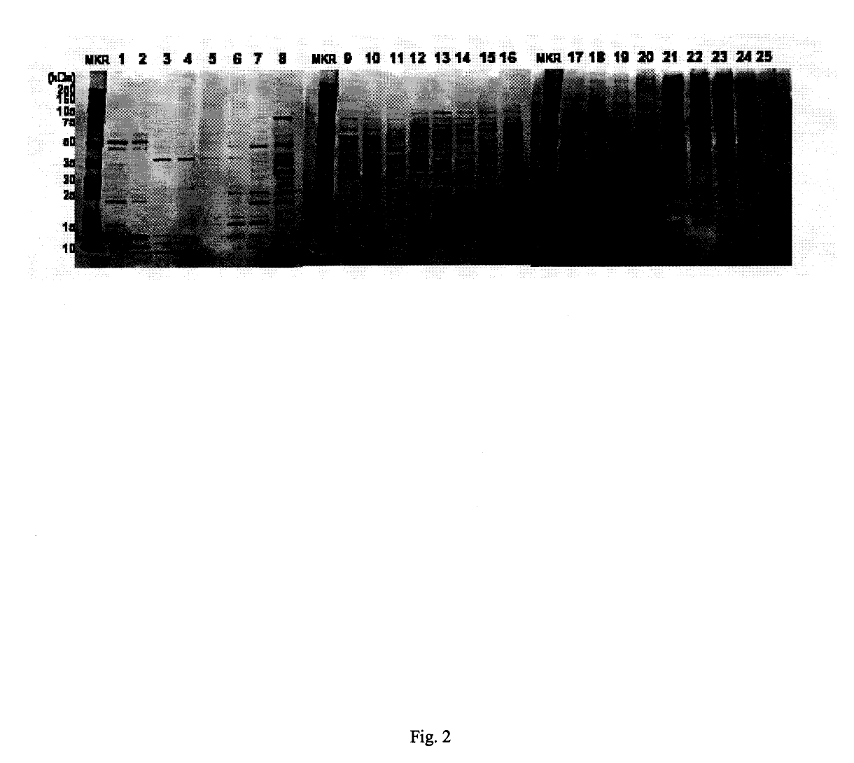

Gels were visualized with traditional Coomassie Blue R250 (FIG. 2), an inexpensive, easy to use stain with fixing conditions and a detection limit inline with subsequent data-dependant MSMS analysis. Stains with greater sensitivity, such as silver and Sypro Ruby, were also used successfully prior to Coomassie staining for detection of less abundant proteins.

FIG. 2 revealed that the initial CAX gradient profile, based on UV absorption at 280 nm, resulted in uneven protein distribution. Protein density was particularly dense toward the end fractions leading to significant band overlap. Based on the protein distribution in FIG. 2, an optimized gradient was generated for differential analysis using a 3 step slope that effectively distributed proteins throughout the available fraction space. FIG. 2 shows rat cerebellum proteome visualized on 1D-PAGE following CAX fractionation. 1 mg of rat cerebellum brain lysate was divided into 25 CAX fractions each resolved further by 1D-polyacrylamide gel electrophoresis (10-20% acrylamide). Protein bands were then visualized by Coomassie blue staining. MKR indicates molecular weight marker lanes.

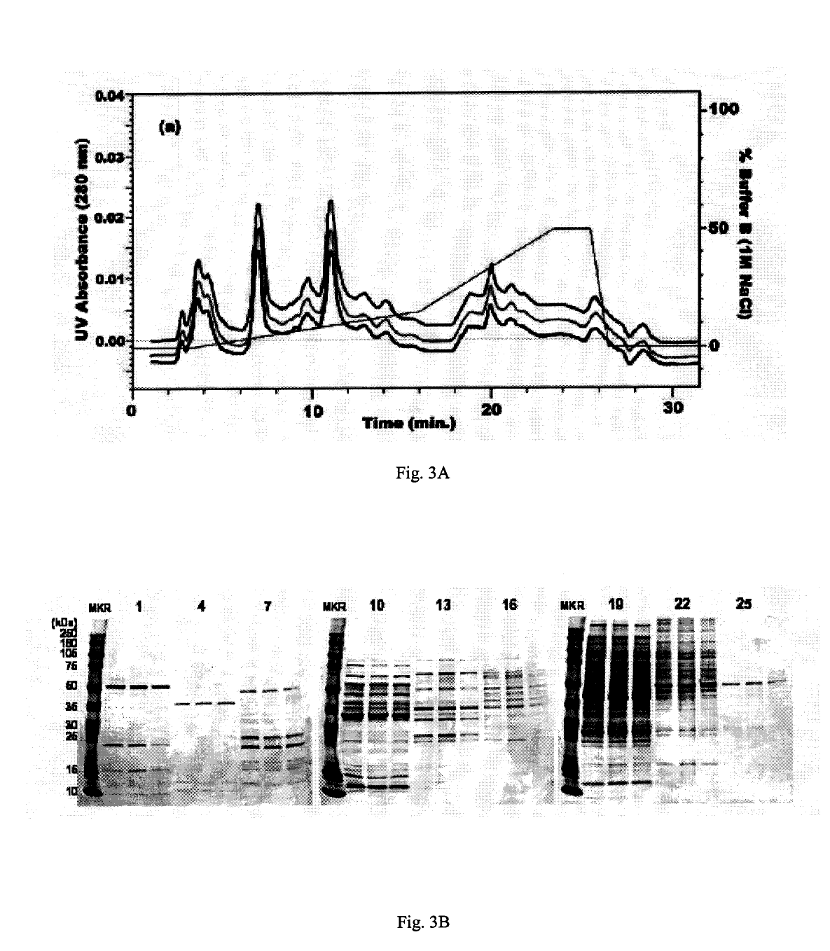

Gel-to-gel reproducibility is a potential problem for all types of protein separations involving PAGE, and a major limitation with differential analysis using 2D-PAGE. CAX-PAGE reproducibility was evaluated after triplicate runs of the same sample. Sequential chromatograms shown in FIG. 3a overlap without significant deviation. Three fraction groups evenly spaced at the beginning (#1, 4, and 7), middle (#10, 13 and 16), and end of the separation (#18, 20 and 24) were loaded in triplicate onto 1D-PAGE (FIG. 3b). Identical protein compliments and an average intensity correlation of 94% (Phoretix 1D software) were observed. The slight variation is primarily attributed to variability in protein recovery from the ultrafiltration and gel loading. This experiment was repeated showing similar run-to-run reproducibility but with a non-uniform shift in retention time when compared with that in FIG. 3. Peak shifting typical of column chromatography occurs as a combination of environmental, buffer, and column aging changes. Samples should be compared running one after another to avoid daily variations.

FIG. 3 shows the reproducibility of CAX-PAGE protein separations. FIG. 3A shows a chromatogram of rat cerebellum brain tissue lysate (1 mg protein) run sequentially in triplicate by CAX. FIG. 3B shows selected fractions (paired as indicated) from the three replicate CAX runs resolved and visualized side-by-side on 1D-PAGE. Protein compliment remained constant while band intensity varied on average by only 6%.

### Example 3

### CAX-PAGE Protein Recovery and Retention

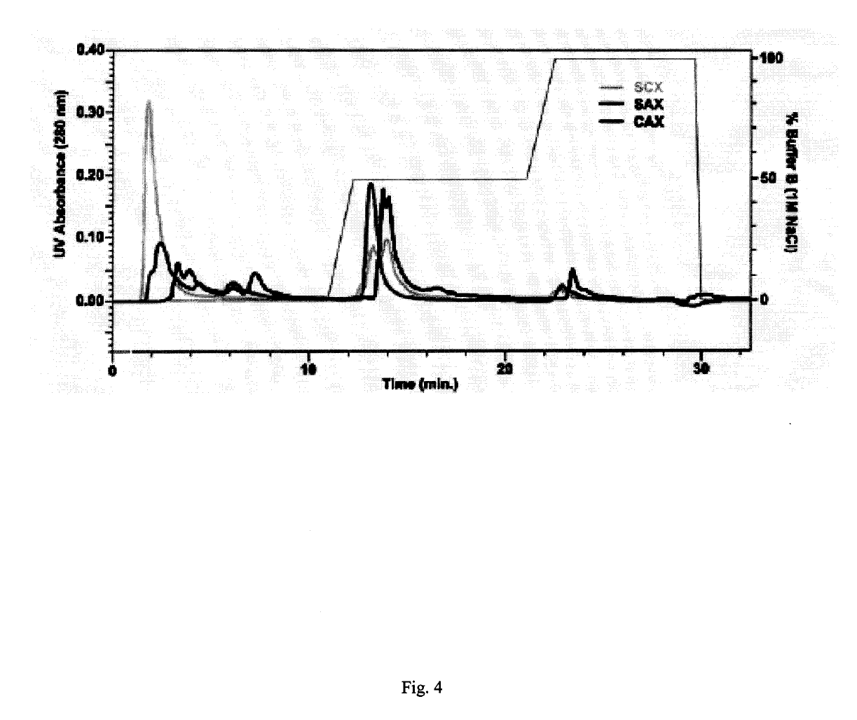

Ion-exchange chromatography has a high loading capacity, making it advantageous as a first dimensional separation. Capacity affords the ability to load a significant amount of protein permitting reasonable sample loss common in multi step processes. Of concern when combining SAX and SCX phases was the possibility of exacerbated protein loss. Protein assay results suggested an increase in protein recovery with CAX at 67% of total protein compared with 49% for separate SAX and 59% for SCX. All assays performed were normalized using a fixed volume of the initial sample; however, fraction composition would differ between separations thereby effecting relative quantitation between ion-exchange modes. In contrast, peak area analysis indicated greater recovery from SCX at an area of 15.5 than for CAX at 12.8 and SAX at 9.25. Both methods suggested SAX may irreversibly trap more than SCX, but no additional sample loss is observed with CAX chromatography. Roughly 60%±10% of the total protein load is recovered, which is reasonable considering the multiple steps required, in particular the ultrafiltration concentration step which is known to exhibit some protein loss. FIG. 4 shows the retention and recovery of CAX-PAGE separation. The results are from a chromatogram of rat cerebellum tissue lysate (750 μg) performed with SCX, SAX, and CAX with two step elution processes.

More telling from this experiment is the increased percentage of protein retained on-column using CAX chromatography. As discussed earlier, a bi-phasic medium will bind positively and negatively charged proteins more efficiently thus increasing bulk retention. So far, 88% of recovered protein is retained by CAX for gradient elution in comparison with 66% for SAX and 47% for SCX as determined by protein assay. The peak area values are similar, with CAX having the largest retained peak area of 10 (84% of total area) compared with 5.2 (55%) for SAX and 5.7 (37%) for SCX. Increased retention, the motivation for CAX, affords the ability to evenly distribute complex protein mixtures across an expandable number of fractions based on gradient optimization.

### Example 4

### Differential Expression Analysis

The potential of CAX-PAGE is realized with its ability to provide differential expression maps for subsequent targeted differential RPLC-MSMS analysis. As a test case, proteomic differences between cerebellum and cortex regions of rat brain were explored. It was expected that the compliment of proteins would be similar in both tissues, but that expression would differ. Clear chromatographic differences in FIG. 5a are observed between cerebellum and cortex lysates sequentially separated by CAX. For differential analysis, fractions from each run are paired and run side-by-side on 1D-PAGE (FIG. 5b), whereby problems of gel-to-gel reproducibility are avoided by always comparing matching fractions on the same gel. Side-by-side fraction pairing as in FIG. 5b allows for direct visualization of differential expression using simple, cost efficient, visible stains (e.g., Coomassie Blue, silver, Deep Purple). Fluorescent stains such as Sypro Ruby also work well, though they require a more expensive fluorescence scanner (three times the cost for the liquid chromatography station). Whether visible or fluorescent stains are used, images are easily assessed with the Phoretix 1D software. Automatic processing is performed to identify gel lanes, providing a clear boundary along the x-axis. Band height is also distinguishable, though fainter bands tend to require manual verification. Band intensity is automatically calculated along with band mass based on calibration with a traditional protein marker. Data is then output to an excel spreadsheet with adjacent bands lined up between lanes. A threshold set at 100% difference in band intensity is applied to generate a list of target bands for further analysis, thereby minimizing mass spectrometry workload in comparison with shotgun proteomic protocols.

FIG. 5 shows a comparison of rat cerebellum and cortex proteomes via sequential CAX and side-by-side 1D-PAGE. FIG. 5A is an overlay of cerebellum and cortex CAX chromatograms at 280 nm. (b) Side-by-side (M=cerebellum on left, X=cortex on right) pairing of 25 fractions run on 1D-PAGE. Boxed bands were excised for protein identification—note letter labeling for correlation with Tables 1 and 2.

### Example 5

### CAX-PA GE Differential Colorization

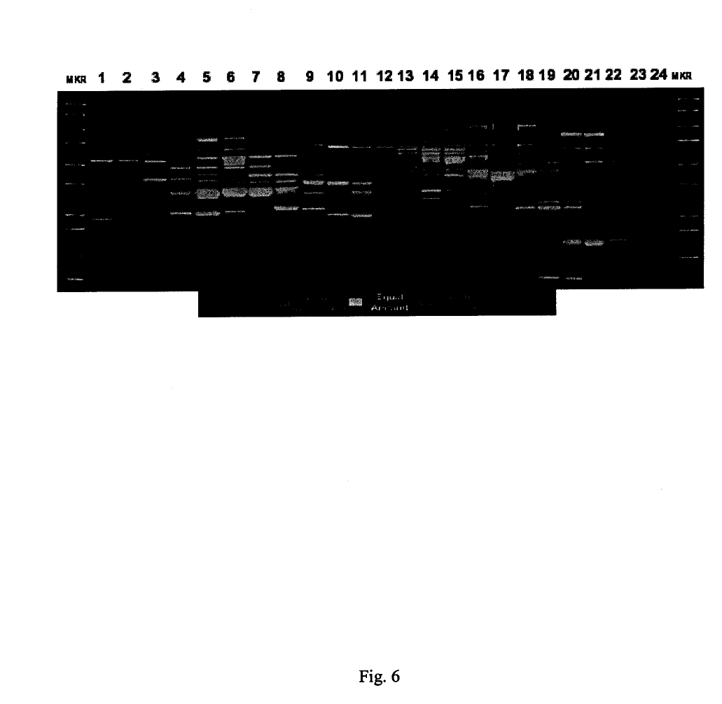

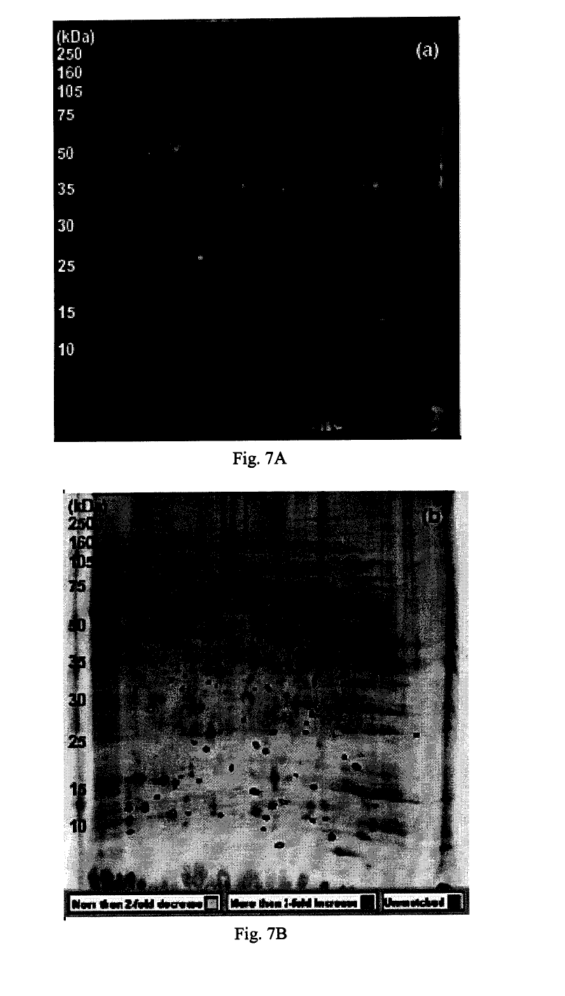

A false-colorization scheme can also be used to aide manual inspection of differential expression, creating images (FIG. 6) similar to those produced with 2D-DIGE (FIG. 7a). The colorized image was generated by converting adjacent cortex and cerebellum lanes into green and red respectively and superimposing the two. A difference in color contrast was not of issue since both colors where generated from the same original grayscale image. Distortion between adjacent lanes was corrected with the rotation and skewing features of Adobe Photoshop to superimpose bands as best as possible. Green represented greater expression in cortex while red emphasized cerebellum. The human eye is adept at recognizing slight color shift (away from yellow at equal expression) more so than recognizing slight changes in grey band intensity. The colorization map was generally used to aide manual confirmation of the Phoretix 1D output as it helped in distinguishing overlapping bands.

FIG. 6 shows a colorized rat cerebellum-cortex differential proteome display after CAX-PAGE. The colorized display was performed by overlaying adjacent lanes from FIG. 5B false colored red for cerebellum and green for cortex. Color aides in manual inspection of the differential separation.

FIG. 7 shows the rat cerebellum-cortex differential proteome display using 2D-DIGE. 2D-DIGE display of the pooled cerebellum and cortex lysates used with CAX-PAGE. FIG. 7A is a false-color overlay of cortex Cy3 (green) and cerebellum Cy5 (red) labeled DIGE images. FIG. 7B show the results of 2D differential software analysis comparing cortex and cerebellum tissue. Spots with 100% difference between samples are indicated by yellow for greater in cortex and green for greater in cerebellum, while blue indicates spots found only in one sample.

### Example 6

### Comparing Differential Analysis by CAX-PAGE and 2D-DIGE

Analysis of the same cortex and cerebellum tissue lysates was performed by 2D-DIGE a prominent alternative method that serves as a reference in determining CAX-PAGE effectiveness for differential analysis. The Cy3 and Cy5 images shown overlaid in FIG. 7A were compared using Phoretix 2D image analysis software with the result illustrated in FIG. 7B. Using 2D-DIGE, 45 spots were discerned as more than twice as prominent in cerebellum and 37 spots were more than twice as prominent in cortex (FIG. 7B) for a total of 82 differential protein targets. In comparison, CAX-PAGE revealed 105 band intensities more than twice as prominent in cerebellum and 41 bands more than twice as prominent in cortex for a total of 146 targets, 78% more than from 2D-DIGE. Proteins of high concentration, which appeared as very large spots with 2D-DIGE also posed a problem for CAX-PAGE. As peak width in CAX chromatography is proportional to protein concentration, highly expressed proteins fell across multiple fractions appearing as dark streaks. RPLC-MSMS analysis confirmed the same protein in each streaked band. Fortunately, few of these streaks are observed with brain tissue lysate. Nine of the 146 band pairs were found to be redundant, reducing total targets to 137, 67% over that observed in 2D-DIGE.

CAX-PAGE provided an increased mass range for differential analysis. Of the 137 differential targets found with CAX-PAGE, 13 were at a mass of 100 kDa or greater. In comparison, no differential targets were uncovered above 100 kDa using 2D-DIGE. The ability to discern differences at high mass is particularly relevant in brain injury paradigms. Cytoskeletal proteins are often of great mass. This protein class is particularly prone to proteolysis associated with neuronal death after brain injury. Thereby exclusion of high mass proteins would unduly eliminate key biomarkers of brain injury.

Signal intensity differed somewhat between the two cyanine dyes giving a bias toward green or red from one gel to the next. As well, more background was detected at the emission wavelength for Cy3 over Cy5, making fainter spots more difficult to discern. Issues of lower than expected sensitivity, migration differences between labeled and unlabeled protein, and handling complexity are well known for 2D-DIGE, though the introduction of saturation 2D-DIGE labels has helped with some of these issues.

In summary, CAX-PAGE generated more differential targets using less expensive more easy to use Coomassie staining and imaging. As many as 13 differential proteins were observed at a mass greater than 100 kDa using CAX-PAGE compared with none for 2D-DIGE. CAX-PAGE also maintains spatial separation of samples, permitting further differential quantitative and qualitative analysis by subsequent RPLC-MSMS.

### Example 6

### Resolving Power of CAX-PAGE

Though CAX-PAGE revealed more differential targets than 2D-DIGE using the same samples, the resolving power of CAX-PAGE as performed here is lower than that of 2D-DIGE and other 2D techniques. The most common means for comparing multi-dimensional separations is the use of theoretical peak capacity (n_{c}). Total n_{c} is essentially the product of individual peak capacities for each dimension of separation. This generally assumes in the case of serial separations, that n_{c} for the first dimension is unaltered by the second separation. For 2D-PAGE in particular, this is not the case since protein spot diffusion occurs in two dimensions after the IPG strip is transferred to SDS-PAGE. Total n_{c} can still be determined based on final spot dimensions (x and y axis width values) divided into the length of separation for each axis. From the 2D-DIGE separation shown in FIG. 7, x-axis n_{c} (mainly IEF) came to be 73.5 and y-axis (SDS-PAGE) n_{c} 74.0. This generates a theoretical total n_{c} of 5440, about average for 2D-PAGE.

CAX-PAGE has an x-axis n_{c} equal to the number of ion-exchange fractions collected, in this case 25 or a third that of IEF, but independent of x-axis band broadening on 1D-PAGE. In contrast, CAX-PAGE has twice the peak capacity along the y-axis at 143, achieved as a result of the larger x-axis width and the stacking gel at the top of the 1D-PAGE (not used with 2D-PAGE). Despite a shorter gel length, the greater y-axis n_{c} of CAX-PAGE partially compensates for the small fraction number producing a total n_{c} of 3570, which is 34.4% shy of that calculated for 2D-DIGE. Peak capacity can also be stated as a working value, which in this case was calculated to include a rectangular separation space beyond which no proteins migrate. Determined from the images shown in FIGS. 5 and 7, the working values for CAX-PAGE and 2D-DIGE are 3120 and 4030 respectively, indicating that the actual resolving power of these techniques are even closer. To improve CAX-PAGE, we recently moved to larger format Protean II Bio-Rad gels (16 cm×16 cm), which come prefabricated with up to 20 wells. Preliminary results show an increased y-axis n_{c} of 211, and along with an expansion of CAX separations to 36 fractions (two gels per sample), provides a theoretical peak capacity of 7600.

### Example 7

### Differential Semi-Quantitation and Protein Identification by Capillary RPLC-MSMS—Third and Fourth Dimensions

A notable advantage of CAX-PAGE, as well as the other novel differential approaches discussed in the previous section, over 2D-DIGE is the maintenance of spatial separation between each sample. This is not possible with 2D-DIGE since inherent to this technique, indeed the driving force behind it, is that samples are mixed together and run simultaneously on the same gel to avoid gel-to-gel variability. Maintaining spatial separation between samples as afforded by CAX-PAGE is essential for further differential analysis. The presented multidimensional protocol involves selection of differential targets identified after CAX-PAGE, excision of these band pairs, digestion with trypsin, and peptide separation using capillary reverse phase liquid chromatography. Separation of tryptic digests is integral to this protocol as sample complexity is further increased by in-gel digestion, and it is often the case that more than one protein is present within the excised band. Peak capacity for capillary RPLC is high due to the enhanced efficiency of small columns, with n_{c} values ranging between 100 and 200. RPLC elutes tryptic peptides spread out in time onto a tandem mass spectrometer, the fourth dimension of separation using mass-to-charge. The peak capacity of a dynamic exclusion MSMS scan method can be calculated as the parent ion scan width (800 m/z) divided by the dynamic exclusion width (3 m/z) resulting in an n of 267. Combining the four separation dimensions provides an immense total n_{c} of 1.43×10⁸. Assuming a peptidome estimate of 5×10⁴ proteins divided into 100 peptides the component number (m) is 5×10⁶. That would indicate a system saturation factor (α) of only 3.50% (α=m/n_{c}). At this α, 93.2% of tryptic peptides would be independently resolved by this protocol using Poisson statistics. In other words, the theoretical peak capacity for this protocol is significantly greater than the assumed component number such that nearly all components will be resolved from one another even though the working peak capacity of the system is considerably less than theoretical.

The power of this multi-dimensional protocol lies in the use of four independent physical properties: 1) protein charge distribution; 2) protein molecular weight; 3) tryptic peptide hydrophobicity; and 4) tryptic peptide mass-to-charge. To proceed, three assumptions were made with regard to the MSMS data. The first was that having been visible with Coomassie staining, a protein would be identified by two or more peptides using strict cross-correlation values, since the detection limit of Coomassie stain and dynamic exclusion MSMS are similar with our instrument. The second assumption is that in order to have produced a band intensity difference of 100% or more between samples the responsible protein would have had a similar or greater expression level relative to background proteins. The last assumption is that only the differentially expressed protein, not equally expressed background proteins, will exhibit a discernable difference in the number of peptides identified between the two samples, taking advantage of the semiquantitative nature of peptide number in bottom-up MSMS analysis.

To evaluate the protocol two protein groups were selected for RPLC-MSMS analysis: (1) a random selection of band pairs from the CAX-PAGE differential target list (those showing a 100% difference in band intensity) as indicated in Table 1; (2) a random selection of band pairs that did not make the differential target list (less than 100% difference in band intensity) as indicated in Table 2. In total, 85% of MSMS runs fulfilled the first assumption irrespective of whether the band was differential or not. The fact that 15% of bands did not reveal obvious identified proteins is not surprising considering that most of these bands tended to be of low intensity. An enhancement in mass spectrometer sensitivity, possible with the new generation of linear ion traps, would improve protein identification determination.

To assess the validity of assumptions two and three, we compared the two groups (Table 1 and 2) as to how often the semi-quantitative result using peptide number either matched, did not match, or was inconclusive when compared to CAX-PAGE band intensity. In the first group, both peptide number and band intensity reflected higher expression in the same tissue 89% of the time. An inconclusive determination from peptide number happened only 7% of the time, with only 1 case (4%) where the two results clearly did not match. The success rate of 89% was in stark contrast with results from group 2, where only 28% of cases resulted in a match of the same tissue. Without a 100% difference in band intensity, the peptide results were just as likely to predict a mismatch (also 28%) in tissue assignment. In 44% of cases a clear difference in peptide number could not be discerned, simply because the identified protein was expressed near equally in both samples. In brief, using the two step differential analysis of the CAX-PAGE/RPLC-MSMS platform will result 76% of the time in a verified definition of a particular protein being expressed more than twice as much in one sample over the other. This value can be improved by using a more sensitive mass spectrometer that would more conclusively identify peptides thereby improving semi-quantitative evaluation and protein identification.

Summarizing the differential findings, differentially identified proteins in Table 1 fit into three distinct protein classes known to be prominent in the brain and listed here in order of prevalence: metabolic enzymes such as alpha enolase, pyruvate kinase 3, transketolase, GMP synthase, fatty acid synthase, etc.; neuronal function proteins such as albumin, calbindin 1 & 2, translin, transferrin, etc.; microtubule proteins such as chloride intracellular channel 4 and MAP2. Proteins were identified over a wide molecular weight distribution from 16 to 273 kDa. This is a notable improvement over 2D-PAGE, which under represents proteins above 120 kDa, and far exceeds the current mass range of top-down mass spectrometry approaches. Another advantage of CAX-PAGE is that hydrophobic membrane proteins are readily soluble in the employed SDS lysis buffer, and should not precipitate out during separation, a known problem with 2D-PAGE. This however was not confirmed here likely because membrane proteins are generally of low concentration and only 53 bands were excised.

TABLE 1
Semi-quantitative results and protein identification of gel band pairs showing greater than
100% difference in intensity between cerebellum and cortex - differential target list.

| Gel Data | | | MSMS Data | | | | | Database Search Results | | |
|---|---|---|---|---|---|---|---|---|---|---|
| Exercised | Gel Band | % M to X | Expressed > | # Peptides | M % as | # Peptides | X % as | MW of ID'd | Rat Protein | |
| Band | MW | Difference^{a} | in MSMS^{b} | in M | Covered | in X | Covered | Protein | Identified | Accession # |
| 2 | 46.3 | 2094% | X | 0 | 0 | 2 | 5.3 | 47.5 | Alpha Enolase | NP_036686.1 |
| | | | | | | | | | (Enolase 1) | |
| 3A | 53.0 | −8256% | M | 12 | 22.2 | 0 | 0 | 57.8 | Pyruvate Kinase 3 | NP_445749.1 |
| 4A | 72.5 | −391% | X | 4 | 5 | 0 | 0 | 76.7 | Transferrin | NP_058751.1 |
| 5A | 148.3 | −365% | M | 6 | 3.5 | 3 | 2 | 180.1 | Amylo-1,6- | XP_342332.1 |
| | | | | | | | | | glucosidense | |
| 6A | 61.3 | −101% | M | 10 | 14.5 | 5 | 8.3 | 71.2 | Transketolase | NP_072114.1 |
| 6C | 15.1 | −288% | M | 3 | 16.2 | 0 | 0 | 15.9 | Coactonin-like 1 | XP_341701.1 |
| 7A | 71.7 | −178% | M | 3 | 4.4 | 0 | 0 | 76.7 | Transferrin | NP_058751.1 |
| — | — | — | M | 2 | 3.6 | 0 | 0 | 70.8 | GMP synthase | XP_215574.2 |
| 10A | 53.8 | −143% | M | 11 | 17.7 | 7 | 13.1 | 57.8 | Pyruvate kinase 3 | NP_445749.1 |
| — | — | — | M | 3 | 5.9 | 0 | 0 | 58 | WD Repent | XP_341229.1 |
| | | | | | | | | | Containing Protein 1 | |
| 10B | 27.5 | −359% | M | 6 | 23.3 | 3 | 14.4 | 31.4 | Calbindin 2 | NP_446440.1 |
| 11A | 88.7 | −168% | M | 3 | 3.5 | 0 | 0 | 95.3 | Trans elongation | NP_058914.1 |
| | | | | | | | | | factor 2 | |
| 11B | 27.4 | −443% | M | 9 | 22.5 | 7 | 29.9 | 31.4 | Calbindin 2 | NP_446440.1 |
| 12A | 61.5 | −634% | M | 6 | 9.1 | 0 | 0 | 68.7 | Albumin | NP_599153.1 |
| 12C | 27.5 | −308% | M | 2 | 10.3 | 0 | 0 | 28.8 | Chloride Intracellular | NP_446055.1 |
| | | | | | | | | | Channel 4 | |
| — | — | — | M | 2 | 8.3 | 0 | 0 | 25.6 | Platelet-activating | NP_446106.1 |
| | | | | | | | | | Factor Acetylhydrolase | |
| — | — | — | M/X | 5 | 16.2 | 5 | 21.4 | 31.4 | Calbindin 2 | NP_446440.1 |
| 13D | 28.1 | −740% | X | 3 | 7.7 | 5 | 19.2 | 31.4 | Calbindin 2 | NP_446440.1 |
| 13E | 26.1 | −1135% | M | 5 | 17.2 | 0 | 0 | 30 | Calbindin 1 | NP_114190.1 |
| 13F | 24.9 | 768% | M/X | 1 | 7.8 | 2 | 13.2 | 23.2 | Rho GDP Dissociation | XP_340776.1 |
| | | | | | | | | | Inhitor alpha | |
| 14A | 222.5 | −2170% | M | 6 | 3 | 0 | 0 | 273 | Fatty Acid Synthase | NP_059028.1 |
| 14B | 100.0 | −186% | M | 3 | 3.7 | 0 | 0 | 105.6 | Hexokinase 1 | NP_036866.1 |
| — | — | — | M | 2 | 1.8 | 0 | 0 | 118 | Insulinase (in rulusin) | NP_037291.1 |
| 14C | 89.2 | −631% | M | 8 | 10.5 | 1 | 1.4 | 96.7 | Brain Glycoprotein | XP_342543.1 |
| | | | | | | | | | Phosphorylase | |
| 14E | 27.5 | −910% | M | 3 | 12.3 | 0 | 0 | 30 | Carbonyl Reductase | NP_062043.1 |
| 14F | 25.8 | −491% | M | 7 | 17.2 | 3 | 11.1 | 30 | Calbindin 1 | NP_114190.1 |
| 15A | 235.9 | −208% | M | 5 | 2.1 | 0 | 0 | 273 | Fatty Acid Synthase | NP_059028.1 |
| 15C | 26.1 | −172% | M | 6 | 27.6 | 3 | 13.4 | 30 | Calbindin 1 | NP_114190.1 |
| 16A | 236.5 | −215% | | | | | | | | |
| 16B | 25.9 | −240% | M | 9 | 21.5 | 3 | 13.4 | 30 | Calbindin 1 | NP_114190.1 |
| — | — | — | M | 2 | 5.4 | 0 | 0 | 30 | Cerebellar Ca-binding | NP_114190.1 |
| | | | | | | | | | Protein | |
| — | — | — | M | 3 | 16.2 | 0 | 0 | 26.2 | Translin | NP_068530.1 |
| 17A | 119.3 | −172% | M | 2 | 1.8 | 0 | 0 | 145.9 | Ca-Dependant | NP_037351.1 |
| | | | | | | | | | Activator Secretion | |
| | | | | | | | | | Protein | |
| 18B | 21.7 | −586% | | | | | | | | |
| 18C | 20.1 | −171% | M | 7 | | 4 | | 27 | TYR 2 Mono- | NP_062249.1 |
| | | | | | | | | | oxygenase (14-3-3) | |
| | | | | | | | | | ζη, θ | |
| — | — | — | M | 2 | 7.3 | 0 | 0 | 30 | Calbindin 1 | NP_114190.1 |
| 19A | 116.4 | −220% | M/X | 0 | 0 | 3 | 2.2 | 198.8 | Microtubule- | NP_037198.1 |
| | | | | | | | | | Associated Protein 2 | |
| — | 150.0 | — | M/X | 3 | 12.6 | 0 | 0 | 42 | Brain Creatin Kinase | NP_036661.2 |
| 19B | 22.7 | 227% | | | | | | | | |
| 19C | 21.6 | −581% | M | 3 | 12.1 | 0 | 0 | 33 | Carbonic Anhydrates 8 | XP_226204.2 |
| 22 | 93.4 | −199% | | | | | | | | |
| 23 | 96.2 | −297% | | | | | | | | |

TABLE 2
Semi-quantitative results and protein identification of gel band pairs
showing less than 100% difference in intensity between cerebellum and
cortex - non-differential target list.

| Gel Data | | | MSMS Data | | | | | Database Search Results | | |
|---|---|---|---|---|---|---|---|---|---|---|
| Exercised | Gel Band | % M to X | Expressed > | # Peptides | M % as | # Peptides | X % as | MW of ID'd | Rat Protein | |
| Band | MW | Difference^{a} | in MSMS^{b} | in M | Covered | in X | Covered | Protein | Identified | Accession # |
| 1A | 25.9 | 64% | M | 5 | 20.7 | 3 | 10.6 | 25.9 | Glutathione | NP_058710.1 |
| | | | | | | | | | S-Transferase | |
| 1B | 24.7 | 81% | M/X | 4 | 15.3 | 5 | 20.7 | 25.9 | Glutathione | NP_058710.1 |
| | | | | | | | | | S-Transferase | |
| 1C | 23.2 | 36% | M/X | 2 | 5.6 | 3 | 10.6 | 25.9 | Glutathione | NP_058710.1 |
| | | | | | | | | | S-Transferase | |
| 1D | 16.4 | 77% | X | 2 | 10.5 | 6 | 25.6 | 22.1 | Peroxinedoxin | NP_446062.1 |
| | | | | | | | | | 5 Precursor | |
| 3B | 39.3 | 90% | M/X | 7 | 18.2 | 6 | 12.8 | 46.3 | Glutamate | NP_036703.1 |
| | | | | | | | | | Oxaloacetate | |
| | | | | | | | | | Transaminase 1 | |
| 4B | 45.6 | −53% | M | 7 | 11.5 | 3 | 7.6 | 47.2 | Alpha Enolase | NP_036686.1 |
| | | | | | | | | | (enolase 1) | |
| 4C | 32.3 | −34% | M | 5 | 21.5 | 3 | 9 | 34.7 | Pyridoxal Kinase | XP_342113.1 |
| 5B | 47.1 | −17% | M/X | 5 | 9.7 | 4 | 7.6 | 47.2 | Alpha Enolase | NP_036686.1 |
| | | | | | | | | | (enolase 1) | |
| 6B | 46.3 | 28% | X | 3 | 7.6 | 5 | 7.6 | 47.2 | Alpha Enolase | NP_036686.1 |
| | | | | | | | | | (enolase 1) | |
| 7B | 47.8 | 70% | X | 3 | 5.3 | 10 | 14.5 | 47.2 | Alpha enolase | NP_036686.1 |
| | | | | | | | | | (enolase 1) | |
| 9A | 34.5 | −85% | X | 3 | 11 | 5 | 15.2 | 39.5 | Aldolase | NP_036629.1 |
| 9B | 27.4 | −32% | X | 2 | 8.3 | 5 | 24.4 | 28.9 | Phosphoglycerase | NP_445742.1 |
| | | | | | | | | | Mutase 1 | |
| 12B | 33.4 | −5% | | | | | | | | |
| 13A | 60.9 | −85% | M/X | 9 | 17.9 | 8 | 16.3 | 68.7 | Albumin | NP_599153.1 |
| 13B | 33.6 | −16% | M/X | 5 | 15.6 | 6 | 18.3 | 36.6 | Lactate dehydrogenase | NP_036727.1 |
| | | | | | | | | | B | |
| 13C | 32.2 | 51% | M | 5 | 16.9 | 3 | 9.5 | 35.6 | Malate Dehydrogenase | NP_112413.2 |
| | | | | | | | | | B | |
| 14D | 33.6 | −73% | M/X | 8 | 20.1 | 7 | 21 | 36.6 | Lactate | NP_036727.1 |
| | | | | | | | | | Dehydrogenase B | |
| 15B | 27.8 | 0% | M | 4 | 14.4 | 1 | 5.9 | 31.4 | Calbindin 2 | NP_446440.1 |
| 17B | 35.5 | −12% | M/X | 7 | 19.1 | 8 | 15.7 | 42 | Brain Creatin Kinase | NP_036661.2 |
| 17C | 29.5 | 10% | | | | | | | | |
| 18A | 34.3 | −67% | | | | | | | | |

^{a}Greater band intensity is indicated as a positive value in Cortex and a negative value in cerebellum.
^{b}M indicates 2 or more peptides found for cerebellum over cortex;
X indicates the opposite;
M/X indicates a 1 or no peptide difference between tissues for that protein.

CAX-PAGE also can include use of an ion-exchange columns with a smaller i.d. to provide an increase in column efficiency and a reduction in fraction size comparable to what can be loaded onto commercial 1D large format gels. This would make CAX-PAGE automation more comparable with liquid phase 2D techniques that use fraction collection between dimensions without further processing. CAX-PAGE immobilizes protein within a gel matrix and affords a convenient means of visible detection with the considerable resolving power offered by 1D-PAGE. High throughput staining, robotic band excision and digestion will add to largescale uses. With robotic digestion, samples are automatically placed into 96 well plates that interface directly with an autosampler for capillary RPLC-MSMS, which itself is automated for data acquisition and database searching.

Preliminary experiments with differential analysis between three different stroke injury conditions have been successful. The platform using the same fraction from each sample is grouped on a single gel (i.e., fraction 1 from each sample on gel 1 etc.). The upper limit to the number of samples is therefore determined by the number of lanes within a single gel (up to 19 samples with large format gels). CAX-PAGE/RPLC-MSMS in comparison with other separations strategies does not provide a direct evaluation of isoelectric point (pI). This bit of information is particularly useful when spot location on a 2D map is employed for protein identification as often done with 2D-PAGE; however, pI as determined by isoelectric focusing, free-flow electrophoresis or chromatofocusing is also an additional means to confirm protein identity as determined by mass spectrometry methods. 2D chromatography employing these separations on the other hand does not predict protein mass. Instead unpredictable hydrophobicity is used, which forgoes use of 2D map databases for protein identification. Protein mass is used with CAX-PAGE as a secondary confirmation of protein identity. Additionally, a preliminary investigation showed a correlation between CAX elution and pI. Though the precision of this indirect relationship is low, foreseeably, CAX fraction could be used for validation purposes even if not for assigning pI. In application of CAX-PAGE, mass validation is performed in light of possible protein degradation, common after brain injury.

Differential protein expression analysis allows scientists to map relevant cellular or tissue changes in response to development, environmental stimulus, injury, or disease. The complexity of biological samples has driven the means to resolve and quantify the resulting proteome by use of high resolution separation techniques. A novel 4D approach was presented based on combining bi-polarity ion exchange chromatography in tandem with gel electrophoresis for protein separations followed by capillary reverse phase liquid chromatography online with tandem mass spectrometry for targeted peptide analysis, termed CAX-PAGE/RPLC-MSMS, with a combined theoretical peak capacity of 1.43×10⁸. Straightforward to perform, the platform utilizes traditional visualization stains for cost savings and two complimentary differential determination strategies for validation. The platform was demonstrated for differential analysis between cerebellum and cortex tissues, a test model for biomarker discovery in brain. Using protein separations, 137 distinct targets were revealed out of which 13 had a mass greater than 100 kDa. From the 137 targets, 33 were randomly selected for further peptide analysis by capillary RPLC-MS/MS. Differential expression was confirmed and protein identification was determined in 76% and 85% of cases, respectively. Future efforts are focused on improving chromatographic efficiency for direct coupling with larger format 1D-PAGE. The platform is currently being applied to biomarker discovery for clinical diagnostics of traumatic brain injury, stroke and substance abuse.

### Example 8

### Identification and Preliminary Validation of Novel Biomarkers of Acute Hepatic Ischemia/Reperfusion Injury Using Dual-Platform Proteomic/Degradomic Approaches

To identify proteins differentially displayed in hepatic I/R samples versus control, we examined liver samples using two complementary proteomic techniques: (1) high throughput immunoblotting (HTPI), and (2) combined cation-anion exchange chromatography-sodium dodecylsulphate-polyacrylamide gel electrophoresis (SDS-PAGE)/reversed-phase liquid chromatography tandem mass-spectrometry (CAX-PAGE/RPLC-MS-MS). Based on HTPI, we identified several hepatic proteins that are altered in liver tissue subjected to ischemia/reperfusion injury such as argininosuccinate synthase (ASS), arginase-I (Arg-I), and squalene-synthase (SQS). The most relevant liver-specific proteins identified to date using CAX-PAGE/RPLC-MS-MS are estrogen sulfotransferase (EST), the liver isoform of glycogen phosphorylase (GP), hepatic enolase-1, carbamoyl-phosphate synthetase 1 (CPS-1), and glucose-regulated protein (GRP-58). Preliminary validation of plasma ASS and EST-1 assays demonstrated a greater sensitivity and specificity of these markers for ischemia-reperfusion-induced liver injury as compared with ALT.

Materials and Methods

Rat Model of Ischemia/Reperfusion Injury

All procedures involving animals were performed according to guidelines from the National Institutes of Health and were approved by the IACUC of the University of Florida. Adult male Sprague-Dawley rats (220-250 g) were anaesthetized with 4% isoflurane for 4 min in a chamber until a surgical level of anesthesia was achieved.

Animals were placed on the heating pad and delivery of anesthetic gas continued via a nose cone throughout the surgery. A midline approximately 3-cm-long laparotomy was made, and the liver was exposed. The hepatoduodenal ligament was dissected and occluded for 30 min using an atraumatic vascular clamp. After 30 min of normothermic ischemia, recirculation of the blood through the ischemic liver was achieved by removing the clamp for additional 10, 30 min, 1 and 3 h. At the end of reperfusion, blood was collected from heart; the liver was briefly perfused with cold phosphate-buffered saline (PBS) to remove residual blood and taken for analysis.

Chronic Alcoholic Liver Model

Adult male Sprague-Dawley rats (180-200 g) were kept on nutritionally complete diet containing 36% ethyl alcohol for 14 weeks. An average blood alcohol level (BAL) of 150-175 mg dl⁻¹ was achieved during the experimental period. Blood was collected from the heart; the liver was briefly perfused with cold PBS and taken for analysis.

LPS/D-galactosamine Acute Liver Injury

Lipopolysaccharide (LPS, 011:B5, 50 mg kg⁻¹) plus D-galactosamine (D-Gal, 500 mg kg⁻¹) or saline were injected intraperitoneally (i.p.) in Sprague-Dawley rats as described previously with modifications (Jones et al. 1999. Hepatology 30:714-24; Dokladny et al. 2001. American Journal of Physiology—Regulatory, Integrative and Comparative Physiology 280:R338-R344). Blood was collected 15 min, 45 min, 1 h and 6 h after the treatment.

Liver Tissue Processing and Sample Preparation

Liver specimens were snap-frozen in liquid nitrogen after removal. Liver samples from I/R, naive and sham-operated rats were homogenized on ice using a Polytron in radioimmuno-precipitation assay (RIPA) buffer consisting of PBS, 1% Nonidet P-40, 0.5% sodium deoxycholate, 0.1% SDS, 1 mM DTT, containing 0.1 mg ml⁻¹ PMSF, 1 mM sodium orthovanadate, 5 mM ethylenediamine tetra-acetic acid (EDTA), 5 mM EGTA, and protease inhibitor cocktail (Roche Diagnostics Inc., Indianapolis, Ind.). For r-caspase-3 and r-calpain-2 treatment in vitro, livers obtained from intact (naive) rats were homogenized in RIPA buffer consisting of PBS, 1% Nonidet P-40, 0.5% sodium deoxycholate, 0.1% SDS, 1 mM DTT, 5 mM EDTA, 5 mM EGTA without protease inhibitors and centrifuged for 15 min at 10 000 rpm at 48° C.

Supernatants were removed and the protein was measured using bicinechoninic acid (Pierce). Intact liver samples were treated in vitro with caspase-3 (Chemicon, Temecula, Calif., specific activity 1 mg ml−1) or calpain-2 (Calbiochem, San Diego, Calif., 0.25 mg ml⁻¹) as described previously in detail (Wang et al. 2004 International Reviews in Neurobiology 61:215-240).

High-Throughput Screen Immunoblotting (HTPI)

HTPI is performed as previously described (Yoo et al. 2002, Clinical Cancer Research 8:3910-3921; Malakhov et al. 2003 J Biol. Chem. 278:16608-16613). Briefly, hepatic protein (200 mg) was loaded in one big well across the entire width of the 13×10 cm, 4-15% gradient SDS-polyacrylamide gel (Bio-Rad Criterion IPG, Hercules, Calif.). This translates into approximately 8 mg per lane on a standard ten-well mini-gel. After separation, proteins were transferred to Immobilon-P membrane (Millipore, Billerica, Mass.). The membrane was blocked and clamped with a Western blotting manifold that isolates 40 channels across the membrane. In each channel, a complex antibody cocktail was added and hybridized for 1 h at 37° C. Proteins were visualized with secondary goat anti-mouse antibody conjugated to Alexa680 fluorescent dye (Molecular Probes, Carlsbad, Calif.) and scanned at 700 nm using the Odyssey Infrared Imaging System. Each sample was run twice in two independent experiments. Protein bands from scanned images were measured by densitometry and expressed in arbitrary units. The values of protein bands increased in the I/R samples (up-regulation) were divided by the corresponding numbers in control (sham operated rats) and fold-change is presented as mean±SEM preceded by the ‘+’ sign (Table 3). When the values in the I/R samples decreased compared with control group (down-regulation), then control values were divided by I/R numbers and fold change is presented as mean±SEM preceded by the ‘−’ sign (Table 3). The values>100 indicates that the protein band was present only in the I/R samples (+), or detectable only in control samples (−). The similar calculations were done for caspase-3- and calpain-2-treated liver tissue versus intact samples (Table 4, A and B).

Cation-Anion Exchange Chromatography: SDS-PAGE/Reversed-Phase Lliquid Chromatography Tandem Mass-Spectrometry (CAX-PAGE/RPLC-MS-MS)

The entire experimental procedure is described by Haskins et al. (2004, 2005), Wang et al. (2004, 2005), and Ottens et al. (2005) in detail. Briefly, the LC system is set up to run two columns in line: S-Sepharose and Q-Sepharose. Samples were separated using gradient of mobile phase A (20 mM Tris-HCl) and B (20 mM Tris-HCl containing 1 M NaCl. Fractions were collected, concentrated and subjected to SDS-PAGE on BioRad Criterion Gels, 4-20% Tris-HCl 18 well gels. The samples were run in pairs, sham-operated (control) and I/R. Gels were stained with Coomassie-R250 and protein bands were selected for excision. Band excision, protein reduction, alkylation, digestion and extraction were performed as previously described by us in detail (Haskins et al. 2004; Journal of Neurotrauma 22:629-644, 2005; Wang et al. 2005, Expert Reviews in Proteomics 2:603-614; Ottens et al. 2005, Annals of Chemistry 77:4836-4845). The mass-spectrometry (MS-MS) was performed in a LCQ Deca XP, quadrapole ion trap mass spectrometer. The peptides were loaded on to a reverse phase column and eluted into the MS using an organic gradient and electrospray ionization. Resulting tandem mass spectra were correlated with tryptic peptide sequences extracted from a nonredundant mammalian protein database (NCBI) utilizing the Sequest algorithm (Haskins et al. 2004, Annals of Chemistry 76:5523-5533.2005, Wang et al. 2004; Haskins et al 2005, Ottens et al. 2005). Peptide matches only of high spectral correlation were collected by use of DTASelect software data filtering, and IR versus sham liver proteomes were compared using Contrast software. Specifically, peptide correlation values (X_{corr}) greater than 1.8, 2.5 and 3.5 for singly, doubly and triply charged peptides were selected, respectively. A minimum of two peptides was required for identification.

TABLE 3
Quantitative analysis of proteins and protein breakdown products in liver tissue of
rats subjected to hepatic ischaemia/reperfusion (I/R) compared with control rats.
Liver tissues collected from control rats (n = 4) or I/R-treated rats (n = 4) were pooled.
Proteins were separated by SDS-PAGE and analysed by HTPI using our custom 40
antibody mini-screen in duplicate (Runs 1 and 2) as described in the Materials
and methods in detail. The HTPI images were captured (see FIG. 2, A, B, as
examples) and protein bands were quantified. The values of protein band increased
in the I/R sample (+) were divided by the values in control samples. When the
protein band in the I/R sample was decreased (−), the control sample value was
divided by I/R. The results are presented as mean ± SEM of four independent
measurements.

| | | | | | Direction and fold |
|---|---|---|---|---|---|
| | | | Predicted MW | Observed | change I/R versus |
| Lane | Protein ID | Research area | (intact protein) | MW | control |
| 27 | ASS-24 kDa | mitochondria/urea | 46 | 24 | (+) 14.9 ± 4.5 |
| | | cycle/nitric oxide | | | |
| 27 | ASS-31 kDa | mitochondria/urea | 46 | 31 | (+) >100 |
| | | cycle/nitric oxide | | | |
| 27 | ASS-34 kDa | mitochondria/urea | 46 | 34 | (+) >100 |
| | | cycle/nitric oxide | | | |
| 2 | nNOS type I-67 kDa | nitric oxide synthase | 155 | 67 | (+) 6.2 ± 2.3 |
| 8 | Arginase-I | nitric oxide | 35 | 37 | (−) 1.93 ± 0.2 |
| 9 | Squalene synthase | mitochondria/cholesterol | 48 | 36 | (+) >100 |
| | (SQS) | synthesis | | | |
| 16 | MEK5 | MAP kinase | 50 | 50 | (−) 2.23 ± 0.09 |
| 16 | MEK5 | MAP kinase | 50 | 21 | (−) >100 |
| 12 | β-Catenin | tyrosine kinase | 92 | 92 | (+) >100 |
| 31 | Ninjurin | cell adhesion | 22 | 21 | (−) 19.5 ± 3.9 |
| 13 | α-Actinin | cytoskeleton/cell adhesion | 104 | 113 | (+) >100 |

Results: Identification of Hepatic Proteins Altered During I/R Injury by High-Throughput Immunoblotting (HTPI)

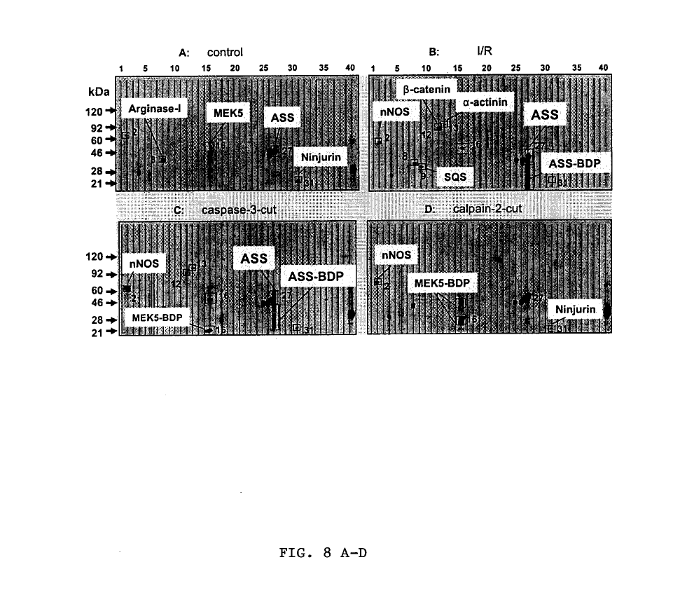

Pooled liver samples from rats subjected to 30 min ischemia followed by 30 min reperfusion and sham operated rats were examined using HTPI. Intact liver tissues were treated in vitro with recombinant caspase-3 or calpain-2 and analyzed by HTPI using the same antibody mini-screen. Initially, we designed a custom mini-array of 40 antibodies from a list of over 1000 antibodies available at BD Pharmingen, (San Jose, Calif.). We selected proteins that are known to be expressed predominantly in the liver and play important roles in hepatic pathophysiology, or are important components of cell cytoskeleton integrity including hepatic cells. The results are presented as images of 40 antibody immunoblotting mini-screen of control (sham-operated) and I/R samples (FIGS. 8A and 8B), in vitro caspase-3-treated intact samples and calpain-2 treated samples (FIGS. 8C and 8D).

Protein bands of interests were quantified in the I/R samples versus sham operated samples, with the results presented in Table 3. The same analysis has been performed for caspase-3- and calpain-2-treated samples versus intact liver tissue (Table 4).

I/R induced up-regulation of various proteins in liver tissue (e.g. ASS, SQS, b-catenin) including a concomitant accumulation of protein breakdown products (e.g. ASS, nNOS), while a modest down-regulation of intact arginase-I was observed without accumulation of detectable cleavage fragments on HTPI. As can be seen from FIGS. 8A-D and Tables 3 and 4, hepatic ASS was up-regulated, predominantly due to a concomitant accumulation of caspase-3, but not calpain-2 mediated cleavage fragments.

In addition to the liver-specific proteins, there was substantial degradation of ninjurin, a non-specific cell adhesion protein, in I/R hepatic tissue compared with sham operated rats (FIGS. 8A and 8B). Ninjurin was degraded mostly via calpain-2-dependent cleavage and, to a lesser extent, via caspase-3 activation (FIGS. 8C and 8D). The p50 MEK5 component of the MAP kinase signaling cascade was significantly degraded, apparently through both caspase-3 and calpain-2 dependent cleavage with the appearance of p21 and p24 fragments, respectively (FIGS. 8A-8D and Tables 3 and 4). Interestingly, β-catenin and α-actinin, both cell adhesion related proteins, accumulated in the liver during I/R, and appeared to be associated with caspase-3 activity (FIGS. 8A-C). Although Arg-I has been considered previously as a potential candidate biomarker of hepatocellular injury, comprehensive studies of its diagnostic value in liver ischemia/reperfusion have not been previously performed. In contrast, ASS and SQS are liver-specific proteins which have not been recognized previously as liver ischemia/reperfusion injury biomarkers.

Analysis of hepatic proteins degraded in response to I/R injury using CAX-PAGE/RPLC-MS-MS: Proteins in control (sham-operated, C) and I/R samples (T) were separated using biphasic ion-exchange chromatography. Protein fractions were collected, paired-up and subjected to SDS-PAGE with Coomassie blue R-250 staining.

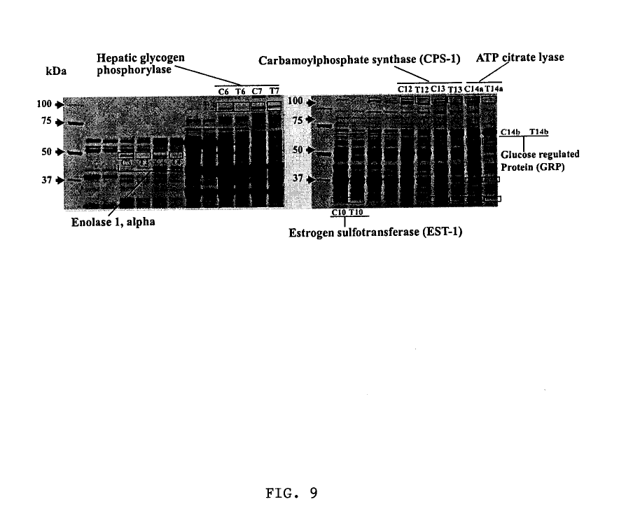

As can bee seen in the differential display (FIG. 9), there are a number of proteins that are degraded in the liver in response to ischemia/reperfusion. A set of down-regulated proteins (greater than a twofold) were selected (squires) after quantification by Phoretix 1D software. Protein bands were excised from control (C) and treated (T) lanes, and digested with trypsin as described in the Materials and methods. Digests were analyzed using reversed-phase liquid chromatography online with tandem mass spectrometry (RPLC-MS-MS). Resulting tandem mass spectra were correlated with tryptic peptide sequences extracted from a non-redundant mammalian protein database (NCBI) utilizing the Sequest algorithm. Peptide matches only of high spectral correlation were collected by use of DTASelect software data filtering, and IR versus sham liver proteomes were compared using Contrast software. Identification and analysis of the most relevant hepatic proteins performed so far are presented in Table 5.

All proteins identified as down regulated in the I/R samples versus controls, exhibited the predicted molecular weight, except carbamoyl-phosphate synthetase 1 (CPS-1), which was identified as 120 kDa protein (predicted 165 kDa) in control samples. Liver glycogen phosphorylase (GP), estrogen sulfotransferase (EST-1), and surprisingly glucose-regulated protein (GRP) demonstrated the highest abundance among degraded proteins. From this list of proteins, CPS-1 has been examined previously as potential biomarker of hepatocellular injury, although comprehensive studies of their diagnostic value in liver ischemia/reperfusion have not been previously performed. The potential diagnostic value of other protein markers, particularly EST-1 and GRP, are until now unknown.

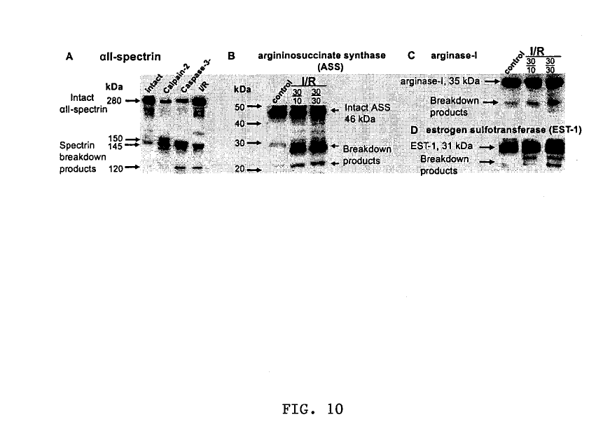

Selection of biomarker candidates and further analysis of their hepatic expression: Next, we selected proteins that were identified by using both HTPI and CAX- PAGE/RPLC-MS-MS for further examination. Based on preliminary data, we selected ASS and EST-1 as lead biomarker candidates for a panel of liver I/R-induced injury. Also, we examined hepatic expression of cytoskeletal protein αII-spectrin and the accumulation of its degradation products (SBDPs) as a ‘standard’ to estimate contribution of caspase-3 or calpain-2 pathways following I/R. Appearance of SBDPs is an established characteristic of aII-spectrin cleavage by caspase-3 (SBDP150i and 120) and/or calpain-2 (SBDP145) in several tissues. Indeed, after 30 min of hepatic ischemia followed by 30 min of reperfusion, expression of intact αII-spectrin (280 kDa) was decreased with concomitant accumulation of spectrin breakdown products SBDP 150i, SBDP145 and SBDP120 (FIG. 10A).

I/R induced a significant increase of ASS breakdown products (approximately 24 and 31 kDa) within 10 min with a further increase at 30 min after initiation of reperfusion (FIG. 10B), consistent with the data obtained using HTPI (FIG. 8B). Hepatic expression of intact arginase-I was not significantly altered 30 min after reperfusion; however, accumulation of protein breakdown fragments (approximately 15-18 kDa) was increased at both 10 and 30 min of reperfuision compared with sham operated rats (FIG. 10C). These products were not displayed on HTPI images, probably due to low molecular weight limitations (FIGS. 8A-D). Surprisingly, there was no significant decrease in intact EST-1 found in the I/R samples within 30 min of reperfuision (FIG. 10D). However, significant amounts of EST-1 breakdown products did accumulate within 30 min after reperfuision (FIG. 10D).

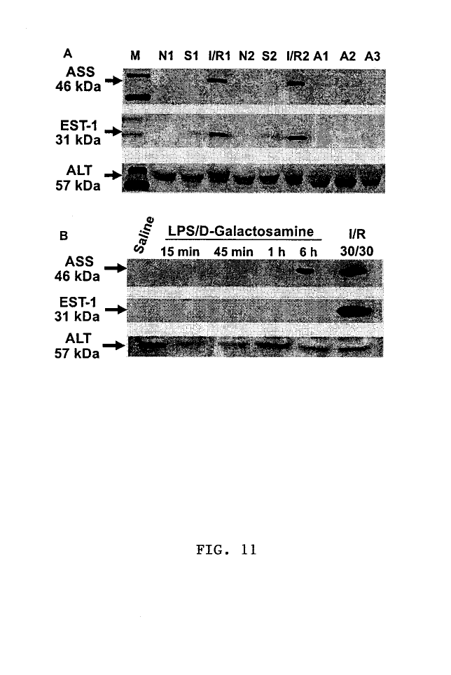

Validation of diagnostic utility of ASS and EST-1: Preliminary validation of diagnostic values of novel hepatic biomarkers was performed by measuring ASS and EST-1 levels in blood after 30 min of reperfusion following 30 min of warm hepatic ischemia in rats. No plasma ASS or EST-1 was detected in intact (N1, N2), sham-operated (S1, S2) or chronic alcohol-treated rats (A1, A2A3) (FIG. 11A). Intact ASS (46 kDa) and EST-1 (31 kDa) proteins accumulated in blood of rats subjected to 30/30 min of warm ischemia/reperfuision (FIG. 11A).

Plasma levels of ALT protein (57 kDa predicted MW) were not changed significantly in I/R and sham-operated rats; in contrast, plasma ALT protein was increased substantially in chronic alcohol rats (FIG. 11A).

Blood levels of ASS, EST-1 and ALT proteins were assessed in rats treated with LPS/D-galactosamine, another model of acute hepatocellular injury. ASS, but not EST-1 accumulated in blood only at 6 hour after i.p. injection of compounds (FIG. 11B). The levels of ALT protein did not change significantly within 6 h after treatment (FIG. 11B).

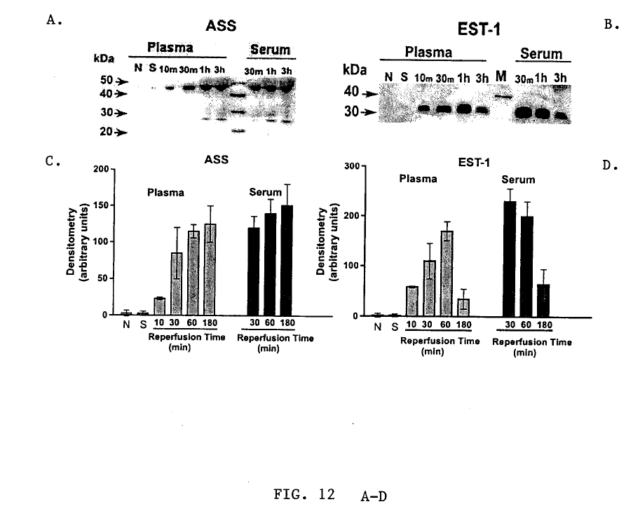

Finally, we examined ASS and EST-1 during time-course of reperffision after 30 min of total ischemia. Blood levels of intact ASS (46 kDa) rapidly attained a steady-state within 30 min, and persisted up until 180 min after initiation of reperffision (FIG. 12A). In contrast, accumulation of ASS breakdown products in circulation (60-180 min) has been delayed compared with liver tissue (FIG. 10B).

Blood levels of EST-1 rose quickly and attained maximum values within 30-60 min followed by a significant decline at 3 h (FIG. 12B). Plasma and serum patterns of ASS exhibited essentially the similar profiles, while serum EST-1 appeared to increase faster than plasma levels suggesting possible contribution of platelet and/or leukocyte EST-1 released into circulation (FIGS. 12C, 12D).

Discussion: For the discovery of novel biomarkers of liver ischemia/reperffision-induced injury, we developed and implemented a liver proteome degradomics approach. Generally, the degradomics methodology is based on the notion that many types of injury to various organs and tissues, including traumatic, ischemic or toxic insult, are mediated via apoptotic and/or necrotic pathways, and as such are accompanied by cleavage (degradation) of several tissue-specific proteins as well as common proteins, such as cytoskeletal αII-spectrin. Thus, identification by differential display of proteins that are degraded (cleaved) in injured tissue versus control reveal molecules, which can be released outside the cells into

TABLE 4
Quantification of proteins and protein breakdown products in normal rat liver
tissue treated in vitro with caspase-3 (A) and calpain-2 (B) related to
untreated controls. Liver tissues from intact rats (n = 4) were digested
in vitro with the recombinant caspase-3 or calpain-2 as described in the
Materials and methods in detail. Proteins were analysed by HTPI using our
custom 40 antibody mini-screen. The samples were run in two independent
experiments in duplicate. The HTPI images were captured (FIG. 2, C, D)
and protein bands were quantified. The analysis and presentation of the
most prominently altered protein bands in caspase-3 digested samples versus
intact liver tissue was performed exactly as described in Table 3.
A

| | | | Predicted | Observed | Direction and fold change |
|---|---|---|---|---|---|
| Lane | Protein ID | Research area | MW | MW | caspase-3 versus control |
| 27 | ASS-21 kDa | mitochondria/urea | 46 | 21 | (+) >100 |
| | | cycle/nitric oxide | | | |
| 27 | ASS-24 kDa | mitochondria/urea | 46 | 24 | (+) 2.57 ± 0.18 |
| | | cycle/nitric oxide | | | |
| 27 | ASS-31/34 kDa | mitochondria/urea | 46 | 31/34 | (+) >100 |
| | | cycle/nitric oxide | | | |
| 2 | nNOS type | nitric oxide synthase | 155 | 67 | (+) 7.73 ± 2.9 |
| | I-67 kDa | | | | |
| 16 | MEK5 | MAP kinase | 50 | 50 | (+) 2.57 ± 0.15 |
| 16 | MEK5 | MAP kinase | 50 | 21 | (+) 8.64 ± 2.66 |
| 12 | β-Catenin | tyrosine kinase | 92 | 92 | (+) >100 |
| 31 | Ninjurin | cell adhesion | 22 | 21 | (−) 3.22 ± 0.43 |

B

| | | | Predicted | Observed | Direction and fold change |
|---|---|---|---|---|---|
| Lane | Protein ID | Research area | MW | MW | calpain-2 versus control |
| 2 | nNOS type | nitric oxide synthase | 155 | 67 | (+) 5.55 ± 2.0 |
| | I-67 kDa | | | | |
| 16 | MEK5 | MAP kinase | 50 | 45 | (+) >100 |
| 16 | MEK5 | MAP kinase | 50 | 24 | (+) >100 |
| 31 | Ninjurin | cell adhesion | 22 | 21 | (−) >100 |

circulation as the full size (intact) proteins and/or in the form of protein breakdown products (BDPs) and serve as potential biomarkers. Specifically, we proposed that proteins subjected to degradation or cleavage, preferentially by activated caspase-3 and/or calpain-2 upon I/R injury and/or protein BDPs, can be accumulated in circulation at early phases of liver damage due to impaired hepatic permeability. Previously, based on degradomics approaches, our group developed HTPI and CAX-PAGE/RPLC-MS-MS and used these techniques for the first time for discovery of biomarkers of traumatic brain injury.

The present studies clearly demonstrate the utility of degradomic approach for development of novel biomarkers of ischemic liver injury. Based on HTPI, we identified several hepatic proteins, which were altered in liver tissue subjected to ischemia/reperfusion injury such as mitochondrial enzymes argininosuccinate synthase (ASS), arginase-I (Arg-I), and squalene-synthase (SQS). Hepatic ASS was up-regulated with concomitant accumulation of caspase-3 mediated degradation fragments, while SQS and Arg-I were up- and down-regulated, respectively, without detectable appearance of breakdown products on mini-screen image.

TABLE 5
Results of CAX-PAGE/RPLC-MS-MS differential analysis of I/R samples versus sham-operated
controls. Select protein bands indicating down regulation (greater than a twofold change) after
I/R were identifed by RPLC-MS-MS. Presented in the table are the protein name, gi reference
number, predicted and observed protein mass, the number of peptides identified (Cₚₑₚ,
Tₚₑₚ) and the percent sequence coverage (C %, T %) for control and I/R samples. Peptide
number is used to validate decreased regulation.

| | Accession | | Predicted | Observed | | | | |
|---|---|---|---|---|---|---|---|---|
| Band | number | Protein | MW | MW | Cₚₑₚ | C % | Tₚₑₚ | T % |
| 4 | gi: 6978809 | enolase 1, alpha | 47.5 | 47 | 4 | 11 | | |
| 6 | gi: 11560087 | liver glycogen phosphorylase | 97.9 | 99 | 2 | 2.1 | | |
| 7 | gi: 11560087 | liver glycogen phosphorylase | 97.9 | 99 | 8 | 17.5 | 2 | 4.8 |
| 10 | gi: 6981594 | estrogen sulfotransferase | 35.4 | 35 | 2 | 7.1 | | |
| 12 | gi: 8393186 | carbamoyl-phosphate synthetase 1 | 164.6 | 120 | 2 | 1.5 | | |
| 13 | gi: 8393186 | carbamoyl-phosphate synthetase 1 | 164.6 | 120 | 3 | 2.3 | | |
| | gi: 8392839 | ATP citrate lyase | 121.5 | 120 | 2 | 2.8 | | |
| 14A | gi: 8393186 | carbamoyl-phosphate synthetase 1 | 164.6 | 120 | 5 | 4.1 | | |
| 14B | gi: 8393322 | glucose regulated protein, 58 kDa | 57 | 56 | 6 | 13.1 | | |

CAX-PAGE/RPLC-MS-MS revealed a number of protein bands down regulated in I/R liver tissue compared with sham operated rats (FIG. 9). Select down regulated protein bands exhibiting a twofold or greater decrease were identified (red boxes) (FIG. 9 and Table 5). Several other protein bands that were also altered significantly (indicated by unlabeled boxes).

While carbamoyl-phosphate synthase (CPS-1) has been reported to be a potentially useful marker of hepatitis, the data regarding enolase-I or liver glycogen phosphorylase (GP) as specific biomarkers associated with hepatic injury are insufficient and controversial. In addition, glucose-regulated protein p58 has been shown to play an important role in toxic liver damage including alcoholic hepatitis, though its significance as potential biomarker has not been studied. In contrast, the roles for estrogen sulfotransferase (EST-1) in hepatic damage including oxidative stress-induced injury were not recognized previously.

Recently, the potential diagnostic value for Arg-I and CPS-1 was reported in rat liver ischemialreperfusion. While Arg-I and CPS-1 appear to be promising candidates as biomarkers for liver I/R injury, the comprehensive studies of these enzymes have not been performed. In contrast, ASS and EST-1 are liver-specific proteins, which have not been recognized previously as liver ischemia/reperfusion injury biomarkers. Our preliminary data indicated that the sensitivity of Arg-1 and, especially CPS-1 is significantly lower than ASS. Thus, ASS and EST-1 were selected for further characterization and validation of novel biomarkers of I/R liver injury.

Caspase-3 and calpain-2 are major executioners of apoptotic and necrotic cell death, respectively, during ischemia or traumatic injury. A signature of caspase-3 and calpain-2 activation in many tissues is a cleavage of several common proteins such as major cytoskeletal αII-spectrin. I/R stimulated a cleavage of αII-spectrin via both caspase-3 and calpain-2 dependent pathways as indicated by accumulation of αII-spectrin degradation products (SBDP). These findings are in great accordance with our previous data on appearance of SBDP in cerebrospinal fluid (CSF) after traumatic brain injury. Conventional Western blot analysis of hepatic ASS expression showed a cleavage pattern upon I/R injury consistent with data obtained using HPTI mini-screen with a detectable accumulation of degradation products within 10 min after reperfusion. Similarly, a slight degradation of hepatic arginase-I and EST-1 was found within the same time-frame following reperffision that was not detected by HPTI due to a lower molecular weight of cleavage fragments.

In the course of research on biomarkers, our laboratories have developed several criteria for biomarker development. Useful biomarkers such as ASS and EST-1 should employ readily accessible biological material such as blood, urine, saliva or CSF, correlate with the magnitude of injury and resulting functional deficits, possess high sensitivity and specificity, have a rapid appearance in biological fluids and be released in a time-dependent sequence after injury. Ideally, biomarkers should employ biological substrates unique to the liver and, at the same time, provide information on injury mechanisms, a criterion that is often used to distinguish biochemical mechanistic markers from surrogate markers of injury since surrogate markers usually do not provide information on injury mechanisms. The use of ASS and EST-1 as biomarkers for liver I/R injury confers a number of important advantages over existing biomarkers. First, ASS and EST-1 proteins are expressed predominantly in the liver and, to a much lesser extent, kidney. Second, ASS and EST-1 are not found in erythrocytes. Thus, assessments of ASS and EST-1 in serum or plasma are not confounded by red blood cell hemolysis. Lastly, ASS is a limiting step in biosynthesis of both urea in the liver and nitric oxide, thereby providing a perfect “pathogenesis-dependent” marker linking ischemia and liver fimction, while EST-1 indicates the conjugative ability of hepatocytes per se.

No blood ASS and EST-1 were detected in intact, sham-operated or chronic alcohol-treated rats, while ASS and EST-1 rapidly accumulated in plasma and serum after 30 min of reperfusion. In contrast, plasma levels of ALT were unchanged during I/R and were significantly elevated in alcohol-treated rats. In the LPS/D-galactosamine-induced model of acute liver damage ASS appeared in blood only 6 h after injection, while after reperfusion following 30-min ischemia, ASS accumulated in circulation within 10 min, rapidly attained a steady state within 30 min, and persisted up until 180 min after initiation of reperfusion. In addition, accumulation of ASS breakdown products in circulation (60-180 min) has been delayed compared with liver tissue. Blood EST-1 was not detectable during 6 h after LPS/D-galactosamine injection, whereas during I/R blood levels of EST-1 rose within minutes and attained maximum within 60 min followed by significant decline at 3 h. Plasma levels of ALT protein were not changed during 6 h after LPS/D-galactosamine treatment, in accordance with previous data showing that serum ALT activity begin to rise at 12 h after treatment peaking at 24 h. Thus, ASS was more sensitive than ALT in detecting of LPS/D-Gal acute liver injury. Hence, its release in blood within minutes (not hours) after I/R indicated higher sensitivity of this marker for detection of I/R-induced liver damage than during acute LPS/D-Gal hepatotoxicity. However, a potential significance of ASS as a marker of other types of acute liver injury requires further investigation.

Based on two platforms of proteomic/degradomic technology, novel biomarkers of liver ischemia/reperfusion induced injury have been discovered. Preliminary validation of the most promising candidates ASS and EST-1 has been performed by measuring blood levels of these proteins and has demonstrated a higher sensitivity and specificity of ASS and EST-1 over established marker of hepatocellular injury ALT. We are currently developing sandwich ELISA diagnostic tests for measurement of ASS and EST-1 in biological fluids. Design of a comprehensive panel of novel and specific biomarkers of I/R injury for subsequent clinical trials will greatly improve diagnostics and management of clinical conditions accompanied by ischemic hepatic darnage. Moreover, further studies of these biomarkers may provide more information on biochemical and molecular mechanisms contributing to liver injury, recovery of function and/or potential targets for novel therapeutic strategies.

### Example 9

### An Improved Design for High Throughput Proteomics Analysis

The experiments were designed to improve CAX chromatography by increasing column efficiency and reducing fraction volume; and to optimize multi-dimensional protein separation of complex brain lysate prior to mass spectrometry analysis.

Materials and methods: we utilized both preparative and analytical sized columns. The standard columns measured 7×35 mm, 50 μm particle size. The Newly test columns were 4×250 mm, 10 μm particle size. The test anion exchange standards used Bio-Rad, Myoglobin, Conalbumin, STI; rat brain lysate.

TABLE 6
Column Performance Stats

| | Myoglobin | | Conalbumin | | STI | |
|---|---|---|---|---|---|---|
| | V_{f} (μl) | V_{f} (μl) | V_{f} (μl) | V_{f} (μl) | V_{f} (μl) | V_{f} (μl) |
| Stnd Column | | | | | | |
| 0.5 ml/min | 167.4 | 125 | 247.8 | 430 | 634 | 352 |
| New Column | | | | | | |
| 0.5 ml/min | 274.5 | 75 | 108.4 | 190 | 158.4 | 130 |

nc = Peak Capacity;
V_{f} (μl) = Fraction Volume

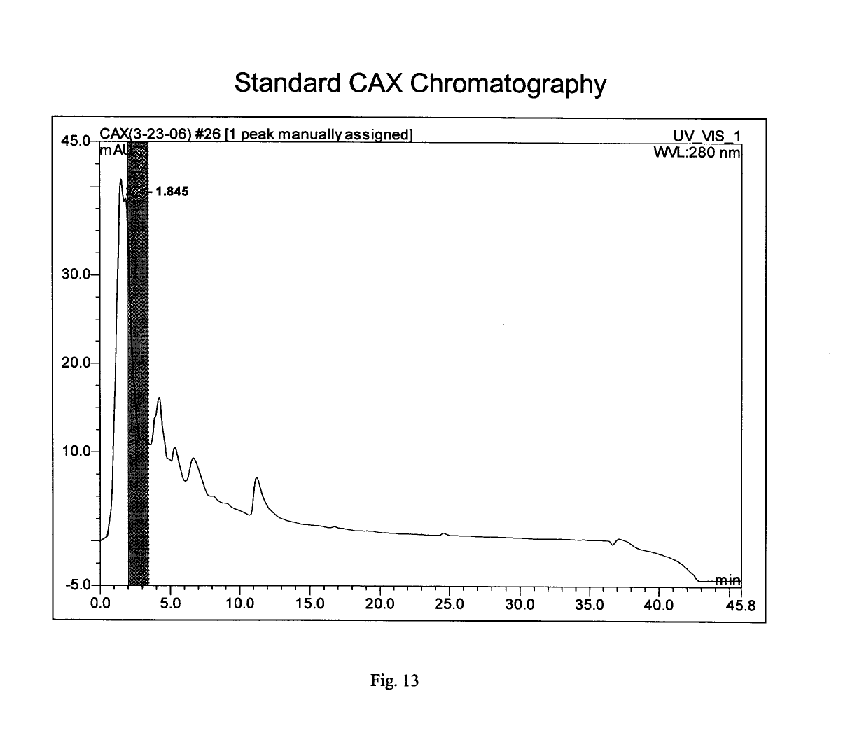

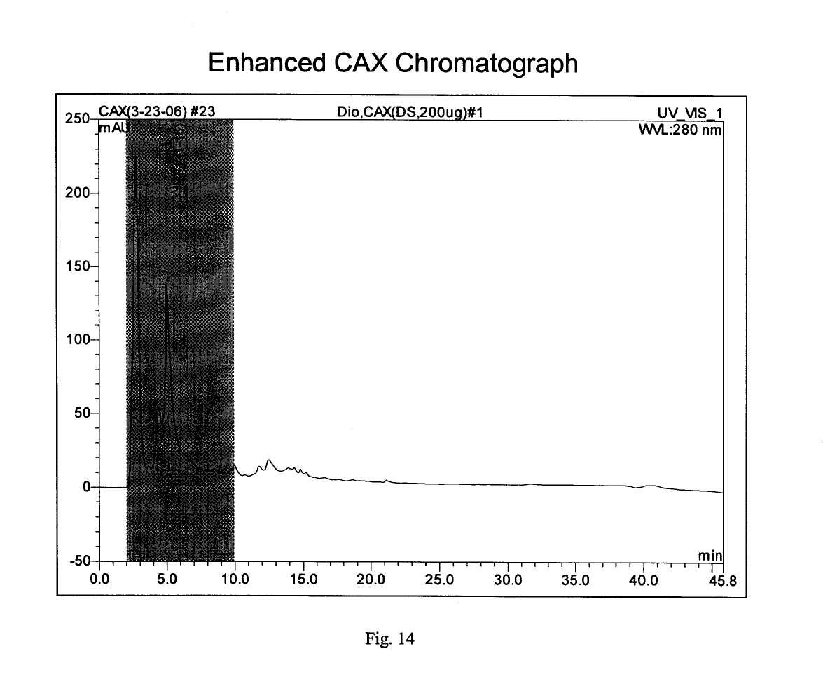

We resolved naïve brain lysate using the standard sized columns for CAX chromatography. 16×200 μL fractions were collected as indicated in the green shaded region, each fraction delineated by a dashed vertical line (See, FIG. 13). The same experiment was repeated with using the newly tested columns in a CAX tandem configuration (FIG. 14).

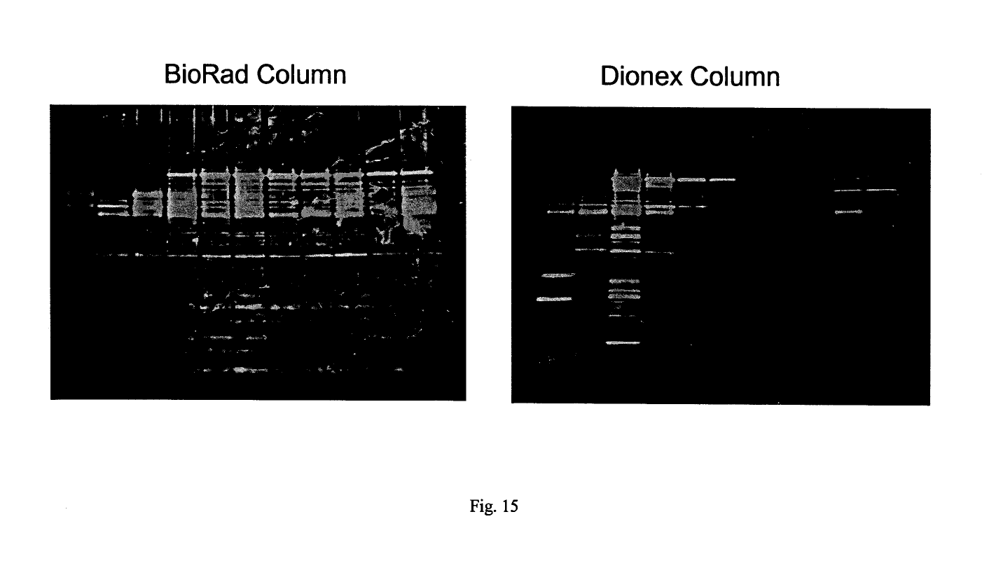

The same brain fraction was resolve by CAX chromatography using the newly tested (longer, smaller bore, smaller particle size) columns in a tandem configuration (cation exchange followed by anion exchange). Again, 16×200 μL fractions were collected at the same point in the salt gradient as with the standard size test (FIG. 13). The data demonstrate that column efficiency is dramatically greater, with proteins resolved into a single 200 μL fraction. This is further illustrated in FIG. 15, where first 11 fractions are resolved by 1D polyacrylamide gel electrophoresis (1D-PAGE). As is seen, the more efficient columns focus proteins into a single 200 μL fraction, rather than spread out over 4 or 5 (˜1 mL). Higher efficiency translates into greater resolution, with reduced fraction volume for easier transfer to 1D PAGE. Our intention is to use this enhanced CAX chromatography to collect between 48 and 96 fractions into a 96-well filtration plate, for either direct digestion and loading onto RPLC-MSMS, or for further resolution by 1D-PAGE. The multidimensional peak capacity is expected to approach between n_{c}=7,000 to 13,000, surpassing 2D-PAGE (between n_{c}=5,000 and 10,000).

By increasing peak capacity we were able to obtain better separation of the rat brain lysate. The results also indicate that when coupled to the second dimension gels the peak capacity of the Newly tested column configuration is greater by two-fold Bio-Rad CAX nₚ=25, Dionex CAX nₚ=50; PAGE nₚ=143. 25×143=3575 vs. 50×143=7150. The decrease fraction volume enables the use of 96-well plates which in turn allows for the use of more high-throughput, robotic devices.

### Other Embodiments

It is to be understood that while the invention has been described in conjunction with the detailed description thereof, the foregoing description is intended to illustrate and not limit the scope of the invention. Other aspects, advantages, and modifications are within the scope of the following claims.

What is claimed is:

1. A method of isolating, quantifying and identifying the biomarker associated peptides, comprising:
obtaining a crude biological sample(s);
clarifying the sample(s) via centrifugation and ultrafiltration;
subjecting the samples sequentially to bi-phasic ion-exchange chromatography and obtaining fractions;
separating fractions by polyacrylamide gel electrophoresis into bands according to molecular weight and quantitatively imaging band density and evaluating protein expression;
cutting selected bands from the polyacrylamide gel and subjecting them to in-gel digestion;
subjecting the digested bands to capillary reverse phase liquid
chromatography in tandem with mass spectrometry; thereby,
isolating, quantifying and identifying the biomarker associated peptides.

2. The method of claim 1, wherein bi-phasic ion-exchange chromatography comprises at least a plurality of gradients.

3. The method of claim 1, wherein the bi-phasic ion exchange chromatography comprises at least a two step gradient.

4. The method of claim 1, wherein the bi-phasic ion exchange chromatography comprises a three step gradient.

5. The method of claim 1, wherein the bi-phasic ion exchange chromatography comprises a five step gradient.

6. The method of claim 1, wherein the bi-phasic ion exchange chromatography comprises a ten step gradient.

7. The method of claim 1, wherein the bi-phasic ion exchange chromatography comprises between about a two step gradient up to a twenty step gradient.

8. The method of claim 2, wherein the two-step gradient comprises a linear transition from 0% to about 15% in a volume of about 12 mL up to 50 mL.

9. The method of claim 3, wherein the three-step gradient comprises a linear transition from about 15% to about 50% in a volume of about 7 mL up to 50 mL, held at about 50% in a volume of about 2 mL up to 50 mL and re-equilibrated to 0% in about 1 mL up to 50 mL volume.

10. The method of claim 1, wherein the bi-phasic ion exchange chromatography comprises a plurality of ion-exchange media.

11. The method of claim 10, wherein the ion-exchange media comprise weak anion and cation exchangers mixed with strong anion and cation exchangers.

12. The method of claim 1, wherein the fractions obtained from the bi-phasic ion-exchange chromatography are concentrated prior to polyacrylamide gel electrophoresis.

13. The method of claim 1, wherein the polyacrylamide gel comprises a gradient of between about 1% up to 50%.

14. The method of claim 1, wherein the polyacrylamide gel comprises a gradient of between about 4% to about 20%

15. The method of claim 1, wherein the polyacrylamide gel is visualized by gel stains.

16. The method of claim 1, wherein bands of proteins and peptides separated on SDS-PAGE gels are quantified by densitometric measurement.

17. The method of claim 16, wherein differentially expressed bands are quantified by densitometric analysis.

18. The method of claim 1, wherein the excised bands are subjected to enzymatic digestion.

19. The method of claim 18, wherein the enzyme digested bands are subjected to reverse phase liquid chromatography.

20. The method of claim 1, wherein n_{c} values of the reverse phase liquid chromatography are between about 100 to about 250.

21. The method of claim 1, wherein fractions eluted from the reverse phase liquid chromatography directly flow into the mass spectrometry and separated by mass-to-charge.

22. The method of claim 1, wherein n_{c} values are at least about 1×10⁵.

23. The method of claim 1, wherein the n_{c} values are about 1×10⁶.

24. The method of claim 1, wherein n_{c} values are about 1×10⁷.

25. The method of claim 1, wherein n_{c} values are about 1×10⁸.

26. The method of claim 1, wherein n_{c} values are about 1×10⁹.

27. The method of claim 1, wherein n_{c} values are about 1×10¹⁰.

28. A method of isolating, quantifying and identifying proteins and/or peptides in complex biological mixtures, said method comprising:
obtaining a crude biological sample(s);
clarifying the sample(s) via centrifugation and ultrafiltration;
subjecting the samples sequentially to bi-phasic ion-exchange chromatography and obtaining fractions;
separating fractions by polyacrylamide gel electrophoresis into bands according to molecular weight and quantitatively imaging band density and evaluating protein expression;
cutting selected bands from the polyacrylamide gel and subjecting them to in-gel digestion;
subjecting the digested bands to capillary reverse phase liquid chromatography in tandem with mass spectrometry; thereby,
isolating, quantifying and identifying the proteins and/or peptides.

29. The method of claim 28, wherein bi-phasic ion ion-exchange chromatography comprises at least a plurality of gradients.

30. The method of claim 28, wherein the bi-phasic ion exchange chromatography comprises at least a two step gradient.

31. The method of claim 28, wherein the bi-phasic ion exchange chromatography comprises a three step gradient.

32. The method of claim 28, wherein the bi-phasic ion exchange chromatography comprises a five step gradient.

33. The method of claim 28, wherein the bi-phasic ion exchange chromatography comprises a ten step gradient.

34. The method of claim 28, wherein the bi-phasic ion exchange chromatography comprises between about a two step gradient up to a twenty step gradient.

35. The method of claim 28, wherein the bi-phasic ion exchange chromatography comprises a plurality of ion-exchange media.

36. The method of claim 35, wherein the ion-exchange media comprise weak anion and cation exchangers mixed with strong anion and cation exchangers.

37. The method of claim 28, wherein the fractions obtained from the bi-phasic ion-exchange chromatography are concentrated prior to polyacrylamide gel electrophoresis.

38. The method of claim 28, wherein the polyacrylamide gel comprises a gradient of between about 1% up to 50%.

39. The method of claim 28, wherein the polyacrylamide gel comprises a gradient of between about 4% to about 20%

40. The method of claim 30, wherein the two-step gradient comprises a linear transition from 0% to about 15% in a volume of about 12 mL up to 50 mL.

41. The method of claim 31, wherein the three-step gradient comprises a linear transition from about 15% to about 50% in a volume of about 7 mL up to 50 mL, held at about 50% in a volume of about 2 mL up to 50 mL and re-equilibrated to 0% in about 1 mL up to 50 mL volume.

42. The method of claim 28, wherein the polyacrylamide gel is visualized by gel stains.

43. The method of claim 24, wherein bands of proteins and peptides separated on SDS-PAGE gels are quantified by densitometric measurement.

44. The method of claim 43, wherein differentially expressed bands are quantified by densitometric analysis.

45. The method of claim 28, wherein the excised bands are subjected to enzymatic digestion.

46. The method of claim 45, wherein the enzyme digested bands are subjected to reverse phase liquid chromatography.

47. The method of claim 28, wherein n_{c} values of the reverse phase liquid chromatography are between about 100 to about 250.

48. The method of claim 28, wherein fractions eluted from the reverse phase liquid chromatography directly flow into the mass spectrometry and separated by mass-to-charge.

49. The method of claim 28, wherein n_{c} values are at least about 1×10⁵.

50. The method of claim 28, wherein the n_{c} values are about 1×10⁶.

51. The method of claim 28, wherein n_{c} values are about 1×10⁷.

52. The method of claim 28, wherein n_{c} values are about 1×10⁸.

53. The method of claim 28, wherein n_{c} values are about 1×10⁹.

54. The method of claim 28, wherein n_{c} values are about 1×10¹⁰.

55. A method of isolating and differential quantitative analysis of proteins and/or peptides in complex biological mixtures, said method comprising:
obtaining a crude biological sample;
subjecting the sample to a bi-phasic ion-exchange chromatography and obtaining fractions;
running the fractions obtained in order of elution side-by-side on a polyacrylamide gel electrophoresis allowing for differential comparison;
quantifying bands obtained by polyacrylamide gel electrophoresis by densitometric scanning;
selecting bands which are differentially expressed at least about two-fold as compared to a normal control;
digesting the differentially expressed bands with enzyme;
subjecting the enzyme digested bands to capillary reverse phase liquid chromatography online in tandem with mass spectrometry; thereby,
isolating and quantifying the isolated proteins and/or peptides.

56. The method of claim 55, wherein differential expression of bands on the polyacrylamide gel are validated by comparing peptide quantity difference with gel band density differences.

57. The method of claim 55, wherein bi-phasic ion-exchange chromatography comprises at least a plurality of gradients.

58. The method of claim 55, wherein the bi-phasic ion exchange chromatography comprises at least a two step gradient.

59. The method of claim 55, wherein the bi-phasic ion exchange chromatography comprises a three step gradient.

60. The method of claim 55, wherein the bi-phasic ion exchange chromatography comprises a five step gradient.

61. The method of claim 55, wherein the bi-phasic ion exchange chromatography comprises a ten step gradient.

62. The method of claim 55, wherein the bi-phasic ion exchange chromatography comprises between about a two step gradient up to a twenty step gradient.

63. The method of claim 55, wherein the bi-phasic ion exchange chromatography comprises a plurality of ion-exchange media.

64. The method of claim 55, wherein the ion-exchange media comprise weak anion and cation exchangers mixed with strong anion and cation exchangers.

65. The method of claim 55, wherein the fractions obtained from the bi-phasic ion-exchange chromatography are concentrated prior to polyacrylamide gel electrophoresis.

66. The method of claim 55, wherein the polyacrylamide gel comprises a gradient of between about 1% up to 50%.

67. The method of claim 55, wherein the polyacrylamide gel comprises a gradient of between about 4% to about 20%

68. The method of claim 55, wherein the polyacrylamide gel is visualized by gel stains.

69. The method of claim 55, wherein the bands are digested by enzymes selected from the group consisting of hydrolases, esterases, carbohydrases, nucleases, deaminases, amidases, proteases, hydrases, fumarase, enolase, aconitase carbonic anhydrase, oxidases, dehydrogenases; transglycosidases; transphosphorylases phosphomutases, transaminases; transmethylases, transacetylases, desmolases, isomerases; and ligases.

70. The method of claim 69, wherein the enzyme is a tryptase.

71. The method of claim 55, wherein the enzyme digested bands are subjected to reverse phase liquid chromatography.

72. The method of claim 55, wherein n_{c} values of the reverse phase liquid chromatography are between about 100 to about 250.

73. The method of claim 55, wherein fractions eluted from the reverse phase liquid chromatography directly flow into the mass spectrometry and separated by mass-to-charge.

74. The method of claim 55, wherein n_{c} values are at least about 1×10⁵.

75. The method of claim 55, wherein the n_{c} values are about 1×10⁶.

76. The method of claim 55, wherein n_{c} values are about 1×10⁷.

77. The method of claim 55, wherein n_{c} values are about 1×10⁸.

78. The method of claim 55, wherein n_{c} values are about 1×10⁹.

79. The method of claim 55, wherein n_{c} values are about 1×10¹⁰.

80. The method of any one of claims 1, 28 or 55, wherein the biomarkers are collected into 96-well plates.
